(19) United States
(12) Patent Application Publication
(10) Pub. No.: US 2025/0349011 A1
(43) Pub. Date: Nov. 13, 2025

(54) IMAGE GENERATION APPARATUS, IMAGE GENERATION METHOD, TRAINING METHOD, AND NON-TRANSITORY COMPUTER-READABLE STORAGE MEDIUM

(71) Applicant: CANON KABUSHIKI KAISHA, Tokyo (JP)

(72) Inventor: HIDEAKI MIZOBE, Kanagawa (JP)

(21) Appl. No.: 19/272,294

(22) Filed: Jul. 17, 2025

Related U.S. Application Data
(63) Continuation of application No. PCT/JP2023/047234, filed on Dec. 28, 2023.

(30) Foreign Application Priority Data
Jan. 20, 2023 (JP) 2023-007580

(51) Int. Cl. G06T 7/00; A61B 3/00; A61B 3/10; A61B 3/12; A61B 6/03; G06T 7/38; G06T 11/00

(52) U.S. Cl. CPC G06T 7/0016; A61B 3/0025; A61B 3/102; A61B 3/1233; A61B 6/032; G06T 7/38; G06T 11/006; G06T 2207/10116; G06T 2207/20081; G06T 2207/30041; G06T 2211/404; G06T 2211/441; G06T 2211/456

(57) ABSTRACT

An image generation apparatus includes an image acquisition unit and an outputting unit. The image acquisition unit acquires a medical image. Based on the medical image acquired by the image acquisition unit, the outputting unit outputs a contrast effect image that depicts a contrast effect corresponding to contrast time that includes contrast time moment that is at least one point in time.

### CROSS-REFERENCE TO RELATED APPLICATIONS

This application is a Continuation of International Patent Application No. PCT/JP2023/047234, filed on Dec. 28, 2023, which claims the benefit of Japanese Patent Application No. 2023-007580, filed on Jan. 20, 2023, both of which are hereby incorporated by reference herein in their entirety.

### BACKGROUND

### Field of the Technology

The present disclosure relates to an image generation apparatus, an image generation method, a training method, and a non-transitory computer-readable storage medium

### Description of the Related Art

In the medical field, a contrast medium that enables image capturing with enhanced visibility of blood flow and the like is sometimes used for acquiring contrast images in a time-lapse manner for diagnostic use, for the purpose of identifying the disease of the subject and/or observing how severe the disease is. Contrast examinations are performed using various imaging apparatuses, such as, for example, fluorescein fundus angiography (FA) examinations using fundus cameras, multi-phase contrast examinations using X-ray computer tomography (CT) imaging devices, Sonazoid-enhanced ultrasound examinations using ultrasound diagnosis devices (echography), and the like. However, while contrast images acquired through a contrast examination are often useful as information for making a diagnosis, some patients may experience severe adverse reactions to a contrast medium, and examinations that involve radiation exposure pose potential health risks. Due to these considerations, contrast-enhanced examinations are limited in frequency or, in some cases, may not be performed even once.

On another front, in recent developments of deep learning technology, a proposal has been made to transform an image in a certain domain into an image in another domain. For example, PTL 1 proposes a method of generating a model configured to, upon receiving an input of a fundus examination image, output an image that reproduces a figure showing an abnormal area. NPL 1 proposes a method of generating a model configured to, upon receiving an input of a retinal fundus photograph not using a contrast medium, output an image that resembles an FA examination image.

### CITATION LIST

### Patent Literature

PTL 1 International Publication No. 2019/142910

### Non Patent Literature

NPL 1 Alireza Tavakkoli, Sharif Amit Kamran, Khondker Fariha Hossain, Stewart LeeZuckerbrod, “A noveldeep learning conditional generative adversarial network for producing angiography images from retinalfundus photographs.”, Sci Rep 10,21580(2020), <https://doi.org/10.1038/s41598-020-78696-2>, made publicly accessible online on Dec. 9, 2020

However, the methods disclosed in PTL 1 and NPL 1 are not enough for desirably acquiring an image that depicts a contrast effect corresponding to contrast time that includes contrast time moment of a certain point in time.

### SUMMARY

The present disclosure is directed to providing a scheme that makes it possible to desirably acquire an image that depicts a contrast effect corresponding to contrast time that includes contrast time moment of a certain point in time.

An image generation apparatus according to a certain aspect of the present disclosure includes: at least one processor; and at least one memory storing instructions, when executed by the at least one processor, causing the image generation apparatus to operate as: an image acquisition unit configured to acquire a medical image; and an outputting unit configured to output a contrast effect image that depicts a contrast effect corresponding to contrast time that includes contrast time moment, the contrast time moment being at least one point in time, based on the medical image acquired by the image acquisition unit, by using an image generation model configured to receive an input of the medical image and generate the contrast effect image depicting the contrast effect.

An image generation apparatus according to another aspect of the present disclosure includes: at least one processor; and at least one memory storing instructions, when executed by the at least one processor, causing the image generation apparatus to operate as: a training unit configured to train, by using training data that includes a medical image group, a contrast image group related to the medical image group, and an imaging condition group pertaining to the contrast image group and including contrast time including contrast time moment, the contrast time moment being at least one point in time, when a medical image in the medical image group and the contrast time are inputted, based on the medical image, an image generation model configured to generate a contrast effect image that depicts a contrast effect corresponding to the contrast time.

The present disclosure further encompasses a non-transitory computer-readable storage medium storing a program causing a computer to function as the steps/units of an image generation method, a training method, and the image generation apparatus stated above.

Features of the present disclosure will become apparent from the following description of exemplary embodiments with reference to the attached drawings.

### BRIEF DESCRIPTION OF THE DRAWINGS

FIG. 1 is a diagram illustrating an example of a schematic configuration of an image generation system including an image generation apparatus according to a first embodiment.

FIG. 2 is a diagram for explaining the concept of an image generation model of an outputting unit in the image generation apparatus according to the first embodiment.

FIG. 3 is a diagram for explaining the training of the image generation model of the outputting unit in the image generation apparatus according to the first embodiment.

FIG. 4 is a diagram for explaining the calculation target area of a loss that is calculated when performing the training of the image generation model of the outputting unit in the image generation apparatus according to the first embodiment.

FIG. 5 is a diagram illustrating an example of a GUI screen displayed on a display in the image generation apparatus according to the first embodiment.

FIG. 6 is a flowchart illustrating an example of processing steps in a method of controlling the image generation apparatus according to the first embodiment.

FIG. 7 is a diagram illustrating a first variation example of the first embodiment for explaining a contrast-time-moment-based period (contrast time) during which an FA examination image(s) is recorded, wherein the FA examination image is a moving image included in teacher data that is used when the image generation model is trained.

FIG. 8 is a diagram illustrating the first variation example of the first embodiment, and illustrating an example of a relationship between the contrast effect image that is a moving image outputted by the image generation model and a ground truth image (FA examination image) that is a moving image included in the teacher data.

FIG. 9 is a diagram illustrating a second variation example of the first embodiment, and illustrating an example of an OCTA image and an FA examination image.

FIG. 10 is a flowchart illustrating the second variation example of the first embodiment, and illustrating an example of processing steps in processing for alignment of an OCTA image and an FA examination image.

FIG. 11 is a diagram illustrating an example of a schematic configuration of an image generation system including an image generation apparatus according to a second embodiment.

FIG. 12 is a diagram illustrating an example of a GUI screen displayed on a display in the image generation apparatus according to the second embodiment.

FIG. 13 is a flowchart illustrating an example of processing steps in a method of controlling the image generation apparatus according to the second embodiment.

FIG. 14 is a diagram for explaining the concept of an image generation model of an outputting unit in an image generation apparatus according to a third embodiment.

FIG. 15 is a diagram for explaining the concept of the image generation model of the outputting unit in the image generation apparatus according to the third embodiment.

FIG. 16 is a diagram illustrating the third embodiment, and illustrating an example of periods of presence/absence of left-eye/right-eye FA examination images included in teacher data that is used when the image generation model is trained.

FIG. 17 is a diagram for explaining the training of the image generation model of the outputting unit in the image generation apparatus according to the third embodiment.

FIG. 18 is a flowchart illustrating an example of processing steps in a method of controlling an image generation apparatus according to a first variation example of the third embodiment.

FIG. 19 is a flowchart illustrating a third variation example of the third embodiment, and illustrating an example of processing steps in interpolation image generation processing.

FIG. 20 is a diagram illustrating the third variation example of the third embodiment, and illustrating an example of a period of presence of an FA examination image included in teacher data that is used when the image generation model is trained and a period of absence thereof.

FIG. 21 is a diagram illustrating the third variation example of the third embodiment for explaining an effective pixel area that is common to an immediately-before FA examination image and an immediately-after FA examination image illustrated in FIG. 20.

FIG. 22 is a diagram illustrating the third variation example of the third embodiment, and illustrating an example of a period of presence of an FA examination image included in teacher data that is used when the image generation model is trained and a period of absence thereof.

FIG. 23 is a diagram illustrating the third variation example of the third embodiment for explaining an effective pixel area in a case where the immediately-after FA examination image illustrated in FIG. 22 is the “shot first” FA examination image in an FA examination.

FIG. 24 is a diagram for explaining the concept of an image generation model of an outputting unit in an image generation apparatus according to a fourth embodiment.

FIG. 25 is a diagram illustrating the fourth embodiment for explaining the presence/absence of FA examination images included in teacher data that is used when the image generation model is trained.

FIG. 26 is a diagram for explaining the training of the image generation model of the outputting unit in the image generation apparatus according to the fourth embodiment.

FIG. 27 is a diagram for explaining the training of the image generation model of the outputting unit in the image generation apparatus according to the fourth embodiment.

FIG. 28 is a diagram illustrating an example of a GUI screen displayed on a display in the image generation apparatus according to the fourth embodiment.

FIG. 29 is a flowchart illustrating an example of processing steps in a method of controlling the image generation apparatus according to the fourth embodiment.

FIG. 30 is a diagram for explaining the concept of an image generation model of an outputting unit in an image generation apparatus according to a fifth embodiment.

FIG. 31 is a diagram for explaining the training of the image generation model of the outputting unit in the image generation apparatus according to the fifth embodiment.

FIG. 32 is a flowchart illustrating an example of processing steps in a method of controlling an image generation apparatus according to a sixth embodiment.

FIG. 33 is a diagram illustrating an example of a GUI screen displayed on a display in an image generation apparatus according to a seventh embodiment.

FIG. 34 is a diagram illustrating an example of a schematic configuration of an image generation model generator according to an eighth embodiment.

### DESCRIPTION OF THE EMBODIMENTS

Modes for carrying out the present disclosure (embodiments) will be described below while referring to the drawings. In the embodiments of the present disclosure to be described below, examples will be given with a still picture or a moving picture in a two-dimensional image or a three-dimensional image in mind, whereas, for easier explanation, the drawings contain illustration using a still picture in a two-dimensional image. That is, “image” dealt with by the embodiments of the present disclosure to be described below shall not be construed to be limited to a still picture in a two-dimensional image.

### First Embodiment

First, a first embodiment will now be described.

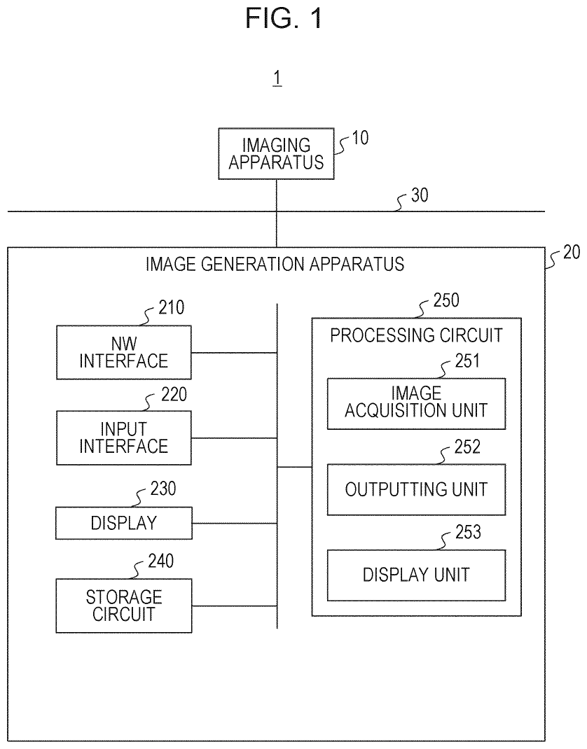

FIG. 1 is a diagram illustrating an example of a schematic configuration of an image generation system 1 including an image generation apparatus 20 according to the first embodiment. As illustrated in FIG. 1, the image generation system 1 includes an imaging apparatus 10, the image generation apparatus 20, and a network 30. The imaging apparatus 10 and the image generation apparatus 20 are connected in such a way as to be able to communicate via the network 30. The schematic configuration of the image generation system 1 illustrated in FIG. 1 is just an example. The number of apparatuses may be modified to any number. In the image generation system 1, an apparatus that is not illustrated in FIG. 1 may be connected to the network 30.

The imaging apparatus 10 is, in the first embodiment, for example, an optical coherence tomography (OCT) imaging apparatus that is capable of picking up an image of the fundus of the subject eye. In the first embodiment, it is sufficient as long as an optical coherence tomography angiography (OCTA) image, which is a medical image derived from OCT imaging, can be acquired at the imaging apparatus 10. Therefore, for example, the imaging apparatus 10 may be replaced with an image management system that stores and manages OCTA images.

As illustrated in FIG. 1, the image generation apparatus 20 includes a network (NW) interface 210, an input interface 220, a display 230, which is a display device, a storage circuit 240, and a processing circuit 250.

The NW interface 210 is connected in such a way as to be able to communicate with the input interface 220, the display 230, the storage circuit 240, and the processing circuit 250. The NW interface 210 controls transfer of various kinds of information and various kinds of data (including image data) to/from each apparatus connected via the network 30, and controls communication therewith. The NW interface 210 is embodied by, for example, a network card, a network adapter, a network interface controller (NIC), etc.

The input interface 220 is connected in such a way as to be able to communicate with the NW interface 210, the display 230, the storage circuit 240, and the processing circuit 250. The input interface 220 converts an input operation received from an operator into an input signal, which is an electric signal, and inputs it into the processing circuit 250, etc. The input interface 220 can be embodied by, for example, a trackball, a switch button, a mouse, a keyboard, etc. Or the input interface 220 can be embodied by, for example, a touch pad on which an input operation is performed by touching an operation surface, a touch screen that includes a touch pad integrated with a display screen, a non-contact input circuit using an optical sensor, a voice input circuit, etc. The input interface 220 is not limited to one that includes physical operation components such as a mouse, a keyboard, and the like. For example, the following constituent entity is also encompassed in the concept of the input interface 220: a constituent entity that receives an electric signal corresponding to an input operation from an external input device provided separately from the image generation apparatus 20 and inputs this electric signal as an input signal into the processing circuit 250, etc.

The display 230 is connected in such a way as to be able to communicate with the NW interface 210, the input interface 220, the storage circuit 240, and the processing circuit 250. The display 230 displays various kinds of information and various kinds of data (including image data) outputted from the processing circuit 250. The display 230 is embodied by, for example, a liquid crystal display, a cathode ray tube (CRT) display, an organic electroluminescent (EL) display, a plasma display, a touch panel, etc.

The storage circuit 240 is connected in such a way as to be able to communicate with the NW interface 210, the input interface 220, the display 230, and the processing circuit 250. The storage circuit 240 stores various kinds of information and various kinds of data (including image data). The storage circuit 240 further stores programs for realizing various functions by being read out and run by, for example, the processing circuit 250. The storage circuit 240 is embodied by, for example, a random access memory (RAM), a semiconductor memory device such as a flash memory, a hard disk, an optical disc, etc.

The processing circuit 250 controls the operation of the image generation apparatus 20 in a central manner, and perform various kinds of processing. As illustrated in FIG. 1, the processing circuit 250 includes an image acquisition unit 251, an outputting unit 252, and a display unit 253. In the present embodiment, a program for implementation of a function as each constituent unit (251 to 253) of the processing circuit 250 is stored in the storage circuit 240 in the form of a computer-executable program. For example, the processing circuit 250 is a processor that implements the function of each constituent unit (251 to 253) by reading the program out of the storage circuit 240 and running the read program. Though it has been explained with reference to FIG. 1 that the processing circuit 250 is a single processor that embodies the image acquisition unit 251, the outputting unit 252, and the display unit 253, a plurality of independent processors may be combined together to constitute the processing circuit 250. In a case where this configuration is adopted, each of the plurality of independent processors constituting the processing circuit 250 may implement the function of the corresponding constituent unit (251 to 253) by running the program.

Though a case where the storage circuit 240 is a single storage circuit has been assumed in FIG. 1, the storage circuit 240 may be split into a plurality of storage circuits. In a case where this configuration is adopted, the processing circuit 250 may read the corresponding program out of each storage circuit and run the read program.

The term “processor” used above may mean, for example, a central processing unit (CPU) or a graphical processing unit (GPU). The term “processor” used above may mean, for example, an application specific integrated circuit (ASIC). The term “processor” used above may mean, for example, a programmable logic device (e.g., simple programmable logic device: SPLD). The term “processor” used above may mean, for example, a complex programmable logic device (CPLD). The term “processor” used above may mean, for example, a field programmable gate array (FPGA). In the present embodiment, the processor implements the function of each constituent unit by reading out, and running, the program stored in the storage circuit 240. Instead of storing the program in the storage circuit 240, the program may be directly integrated in the circuitry of the processor. In this case, the processor implements the function of each constituent unit by reading out, and running, the program integrated in its circuitry.

The image acquisition unit 251 has a function of acquiring a medical image that is a still image of the subject, meaning the target of examination (in the present embodiment, the subject eye), acquired by the imaging apparatus 10. Specifically, the medical image according to the present embodiment is, for example, an OCTA image that is an image of the fundus of the subject eye in fundus examination. The OCTA image will now be described. The OCTA image is an image generated as a blood-vessel image of the fundus of the subject eye by projecting, onto a two-dimensional plane, three-dimensional motion contrast data of the fundus of the subject eye acquired by an OCT apparatus used as the imaging apparatus 10. The motion contrast data is data obtained by taking repetitive image shots, by using an OCT apparatus, of the same cross section of the target of measurement (in the present embodiment, the fundus of the subject eye) and detecting changes over time of the target of measurement between the shots. The motion contrast data is obtained by, for example, calculating, in terms of difference, ratio, correlation, or the like, changes over time in phase, vector, and intensity of complex OCT signals. A two-dimensional enface image of the fundus of the subject eye is generated as an OCTA image by specifying a range in the direction of depth such as a layer in the fundus of the subject eye from the motion contrast data. That is, by specifying one among different depth ranges in the fundus of the subject eye, it is possible to generate an OCTA image in any chosen range, such as a superficial layer, a deep layer, an outer layer, a choroidal vascular network, or the like. The types of an OCTA image are not limited to these examples. OCTA images with different depth range settings may be generated while varying offset values with respect to the layer taken as the reference. In the present embodiment, the description will be given while taking, as examples, an OCTA image in the superficial layer of the fundus of the subject eye and a fluorescein fundus angiography (FA) examination image.

The outputting unit 252 has a function of outputting a contrast effect image that depicts a contrast effect corresponding to contrast time that includes contrast time moment, where the contrast time moment is at least one point in time, based on an OCTA image that is a medical image acquired by the image acquisition unit 251. More particularly, the outputting unit 252 outputs a contrast effect image that corresponds to a still image in a case where the contrast time moment included in the contrast time is a single point in time, and outputs a contrast effect image that corresponds to a moving image comprised of a plurality of still images in a case where the contrast time moment included in the contrast time is a plurality of points in time. In the present embodiment, the outputting unit 252 outputs a moving image as a contrast effect image corresponding to contrast time that includes contrast time moment of a plurality of points in time. Specifically, the contrast effect image according to the present embodiment is a pseudo contrast image that resembles an FA examination image in a moving-picture format depicting time-lapse changes in contrast effect, like those acquired in FA examinations. The outputting unit 252 according to the present embodiment sets, as a play speed of the contrast effect image that is a moving image, a predetermined frame per second (FPS) at which it is easy to observe the change in contrast effect, such as ten frames between seconds. The outputting unit 252 may output the contrast effect image to, for example, the storage circuit 240, or to any other non-illustrated apparatus via the NW interface 210 and the network 30, or to the display 230 concurrently therewith.

The display unit 253 has a function of displaying, on the display 230, the contrast effect image outputted from the outputting unit 252 in such a manner that the operator can observe it easily.

In the present embodiment, the outputting unit 252 includes an image generation model that receives a medical image that is a still image as its input and outputs a contrast effect image that is a moving image that depicts a contrast effect corresponding to contrast time that includes contrast time moment of a plurality of points in time on the basis of the medical image.

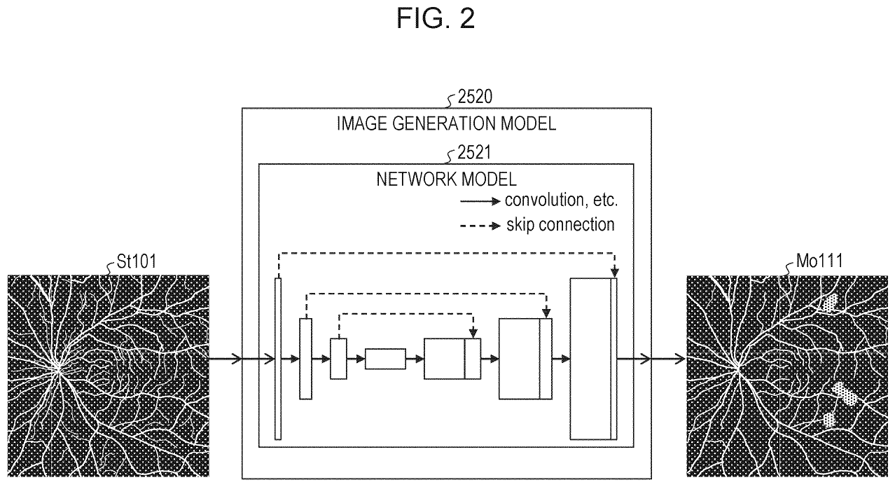

FIG. 2 is a diagram for explaining the concept of an image generation model 2520 of the outputting unit 252 in the image generation apparatus 20 according to the first embodiment.

The image generation model 2520 illustrated in FIG. 2 is a model that includes an image processing system that outputs a contrast effect image by means of, for example, rule-based learning or machine learning (in particular, deep learning technology). In the present embodiment, the image generation model 2520 is a model that has been trained using training data that includes, for example, a medical image group pertaining to medical images, a contrast image group related to the medical image group, and an imaging condition group pertaining to the contrast image group. The image generation model 2520, which includes an image processing system based on deep learning technology, will be described below.

The image generation model 2520 illustrated in FIG. 2 includes a U-Net-based network model 2521 as the image processing system based on deep learning technology. “U-Net” is a known network model using deep learning technology. Specifically, U-Net is trained using a data set comprised of image pairs each of which is made up of an input image and an output image corresponding thereto. When an image is inputted into the image generation model 2520 that includes U-Net having been trained enough, it is possible to output a plausible image corresponding to the input image in accordance with the tendency of the data set that was used for the training. For example, it is known that this can be applied to image segmentation processing, image quality enhancement, image domain transformation, etc. in accordance with the data set.

As illustrated in FIG. 2, the image generation model 2520 inputs an input image St101, which is a still image, after transforming it into a tensor, into the network model 2521, and applies moving-picture transformation to a tensor outputted from the network model 2521 and outputs an output image Mo111. In a case where U-Net is adopted as the network model 2521, there is a need to modify the U-Net. The term “tensor” that appears in the description of the present embodiment means a format expressing a group of pixel values of an image, etc. as a multi-dimensional array; “tensor” is used as form of data input/output to/from the network model 2521; it is assumed that an image and a tensor are mutually transformable.

The following is a specific example. Let us consider a case where the total number of moving-picture frame images of the output image Mo111, which is a moving image that is outputted, is N, and where the shape of the tensor transformed from the input image St101, which is a single still image, is “Cᵢₙ× Hᵢₙ×Wᵢₙ”. In this expression, “Cᵢₙ” denotes the number of channels, “Hᵢₙ” denotes the height of the input tensor, and “Wᵢₙ” denotes the width of the input tensor, where, in particular, the spatial axis of the number of channels may be ignored if “Cᵢₙ” is 1. In the network model 2521 with U-Net modification, the number of elements that constitute the input tensor is increased, and shape deformation is performed up to the last layer, thereby outputting a tensor whose shape is “N×Cₒᵤₜ× Hₒᵤₜ×Wₒᵤₜ”. In this expression, “Hₒᵤₜ” denotes the height of the output tensor, and “Wₒᵤₜ” denotes the width of the output tensor. The tensor outputted from the network model 2521 is divided into N tensors each having a shape of “Cₒᵤₜ×Hₒᵤₜ×Wₒᵤₜ”, and each of the tensors after the division is transformed into a moving-picture frame image. The moving-picture frame images after the transformation are concatenated to be outputted from the image generation model 2520 as the output image Mo111, which is a single moving image. The tensor shape is not limited to the shape described in the present embodiment. It may be any shape with which the same object can be achieved. Though U-Net is taken as an example in the present embodiment, any other network model with which the same object can be achieved may be adopted. Though a two-dimensional image is dealt with in the present embodiment, in a case where a three-dimensional image is dealt with in another embodiment, adding a depth space to the tensor shape described here will suffice.

A data set for training the image generation model 2520, which includes the network model 2521 based on U-Net, will now be described. A data set has a structure of a teacher data group acquired from a plurality of examination targets, wherein an OCTA image that is a still image acquired by taking a shot of the same examination target (that is, the subject eye) and an FA examination image that is a moving image in a predetermined contrast-time-moment-based period (contrast time) are paired to constitute each one piece of teacher data in the group. “Contrast time moment” is moment in time that indicates a lapse from the point in time taken as the reference (the reference point in time) such as the time of administering of a contrast medium to the subject, the time of initial imaging, the time of initial confirmation of a contrast effect on the organ in the acquired image, or the like. “Predetermined contrast-time-moment-based period” (contrast time) is a period defined as in, for example, “from contrast time moment of 0 sec. to contrast time moment of 60 sec.”. In a case where the FA examination image is a moving image of 1 FPS, there exist sixty-one moving-picture frame images corresponding to sixty-one pieces of contrast time moment (i.e., sixty-one points in time) at one-second intervals in the period. A part or the whole of the moving-picture frame images that constitute the FA examination image that is a moving image may be complemented with a still-picture FA examination image.

Depending on the type, settings, etc. of the imaging apparatus 10, it could happen that an FA examination image that is a moving image in a predetermined contrast-time-moment-based period (contrast time) is not comprised of the same number of moving-picture frame images. Therefore, the sampling of the moving-picture frame images is performed so as to make the number of the moving-picture frame images that constitute the FA examination image that is a moving image included in each piece of teacher data uniform among the pieces of teacher data. As a result of performing the above sampling as needed, the FA examination image that is a moving image included finally as a constituent of the data set is comprised of the moving-picture frame images whose number is uniform. When this is performed, the number of said moving-picture frame images agrees with the number of the moving-picture frame images of the contrast effect image that is a moving image outputted by the image generation model 2520.

Depending on the configuration of the network model 2521, sometimes a better result will be obtained if an input image and a ground truth image in teacher data are aligned. Specifically, in the network model 2521 based on U-Net, it is desirable if the OCTA image of the input image in the teacher data acquired by imaging the same examination target is aligned with each of the moving-picture frame images that constitute the FA examination image that is the ground truth image. If, for example, alignment is performed anatomically as this alignment through manual image retouching, image registration processing, or the like, the manner of depicting the contrast effect by the contrast effect image outputted by the image generation model 2520 will become closer to a real FA examination image. Since the OCTA image and the FA examination image are images acquired by imaging apparatuses of different types, their manners of depicting are widely different from each other, and, depending on conditions such as contrast time moment, it is sometimes difficult to perform alignment anatomically. In such a case, first, among the pairs of the OCTA image and the moving-picture frame image group constituting the FA examination image, with regard to at least one pair for which it is relatively easy to perform anatomical alignment, the moving-picture frame image is deformed to perform alignment while referring to the anatomical position of the OCTAimage. Next, while referring to the anatomical position of the moving-picture frame image having been deformed, the rest of the moving-picture frame image group are deformed to perform alignment. Even in a situation where it is difficult to perform anatomical alignment of the OCTA image and the FA examination image, the above procedure makes it possible to perform better anatomical alignment. As a result, the manner of depicting the contrast effect by the contrast effect image outputted by the image generation model 2520 becomes closer to a real FA examination image.

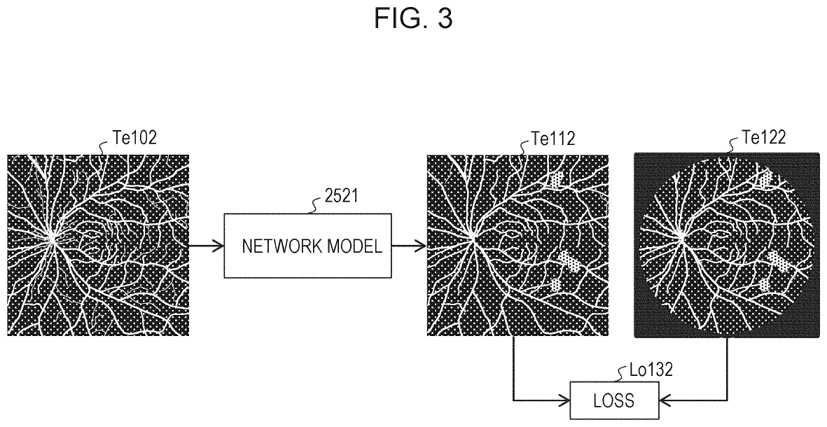

FIG. 3 is a diagram for explaining the training of the image generation model 2520 of the outputting unit 252 in the image generation apparatus 20 according to the first embodiment. In FIG. 3, the same reference signs are assigned to components, etc. that are the same as those illustrated in FIG. 2, and a detailed explanation thereof is omitted. With reference to FIG. 3, the training of the image generation model 2520 using certain one pair of teacher data, that is, processing for updating parameters that constitute the network model 2521 included in the image generation model 2520, will now be described.

First, in FIG. 3, an input tensor Te102, which is a tensor transformed from the OCTA image included in the teacher data, is inputted into the network model 2521. Upon the input, an output tensor Te112, which corresponds to the moving-picture contrast effect image, is outputted from the network model 2521. Next, the image generation model 2520 calculates a loss Lo132, which is an error of the output tensor Te112 compared with a ground truth tensor Te122, which is a tensor transformed from the FA examination image that is a moving image included in the same teacher data. Finally, the image generation model 2520 updates the parameters that constitute the network model 2521 in such a way as to make the loss Lo132 small. This series of update processing is repeated while using a teacher data group assigned for training among the data set until the network model 2521 becomes trained enough. Though an example using a single pair of teacher data for execution of update processing once has been described here for the purpose of explanation, a plurality of pairs of teacher data may be used for execution of update processing once for the purpose of making the learning time shorter, for the purpose of making the learning processing stable, or the like. The learning processing may be aborted in the middle of the learning processing (early stopping) by determining that the accuracy of image generation is high enough in a case where the image generation model 2520 has been trained enough, by performing precision evaluation using teacher data for verification, etc.

A calculation method based on the following approaches can be adopted for precision evaluation and error (loss) calculation between the FA examination image in the teacher data assigned for training or verification (or its tensor) and the contrast effect image outputted by the image generation model 2520 (or its tensor). Specifically, for example, a method of numerically expressing an error or a degree of similarity by using a mean squared error (MSE), a structural similarity (SSIM), or the like can be used. Since precision evaluation and error (loss) calculation are performed on a moving image here, the calculation method based on MSE, SSIM, or the like is used either in a moving-picture-oriented manner or in a still-picture-oriented manner. A manner of performing calculation for a multi-dimensional array of “width× height×time” of a moving image is conceivable as the moving-picture-oriented manner. A manner of calculating an average of results obtained for a multi-dimensional array of “width× height” of moving-picture frame images that constitute a moving image is conceivable as the still-picture-oriented manner. The calculation target in precision evaluation and error (loss) calculation in the training of the image generation model 2520 may be selected while taking, into consideration, a semantic area, which is an area in an image included in training data and is an area that can be demarcated in accordance with the manner of depiction in the image or in accordance with information related to the image. Specifically, the semantic area encompasses a masked area and a non-masked area depicted in the image included in the training data, a printed area containing patient information or imaging information (date and time, imaging protocol name, etc.), and an area indicating an anatomical region or conditions of the organ (normal tissue, abnormal tissue, hemorrhage, inflammation, a white spot, a treatment scar, etc.). In addition, the semantic area encompasses a bright area or a dark area in the image included in the training data, a high-quality area or a low-quality area, and an area where image processing such as alignment has succeeded or failed. As described here, the semantic area is an area in an image included in training data and is an area that can be demarcated in accordance with a manner of depiction in the image or in accordance with information related to the image. For example, in a fundus photograph or an FA examination image acquired by a fundus camera, a masked area (an area blacked out, etc.) could be depicted at the periphery of the image, depending on an imaging angle of field. Since the masked area is an area where the organ is not displayed (an area that has no influence on making a diagnosis), in the training of the image generation model 2520, a non-masked area only, which has an influence on making a diagnosis, may be selected as the target of precision evaluation and error (loss) calculation, and the performance and characteristics of the image generation model 2520 may be adjusted for it.

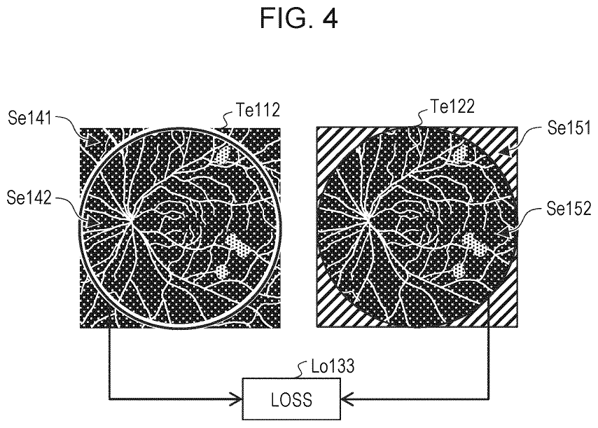

FIG. 4 is a diagram for explaining the calculation target area of a loss that is calculated when performing the training of the image generation model 2520 of the outputting unit 252 in the image generation apparatus 20 according to the first embodiment. In FIG. 4, the same reference signs are assigned to components, etc. that are the same as those illustrated in FIG. 3, and a detailed explanation thereof is omitted.

For example, as illustrated in FIG. 4, sometimes a masked area Se151 could be depicted in an FA examination image. The masked area Se151 may be excluded from the target in performing precision evaluation and error (loss) calculation. Note that, in performing precision evaluation and error (loss) calculation among a plurality of images while taking a semantic area into consideration, if a calculation method of taking a difference at certain pixels located at the same coordinates among the images into consideration is employed, such as in MSE, the pixel area of the target of calculation should be made common among the plurality of images. A specific explanation will be given below while referring to FIG. 4. When the calculation of a loss Lo133 is performed, a non-masked area Se152 of the ground truth tensor Te122 of the FA examination image, and an area Se142, which is included in the output tensor Te112 of the contrast effect image and corresponds to the non-masked area Se152 in terms of coordinates, are designated as the calculation target area.

In a case where the image that is the target of precision evaluation or error (loss) calculation is a moving image, sometimes the position or type of a semantic area varies from one to another of moving-picture frame images that constitute the moving image. Therefore, the method of precision evaluation and error (loss) calculation, and the calculation target area, may be changed from one to another of the moving-picture frame images correspondingly. In particular, if the non-masked area Se152 only is designated as the target when calculating the loss Lo132 for updating the parameters that constitute the network model 2521, the depicting corresponding to the masked area Se151 will be lost in the contrast effect image outputted by the image generation model 2520. That is, since the contrast effect will be depicted in an area Se141, too, the contrast effect about the entire area depicted in the OCTA image inputted into the image generation model 2520 will be observable in the contrast effect image. Conversely, by taking the semantic area out of consideration, the depicting corresponding to the masked area Se151 may be performed to present, to the operator, an image that is closer to a real contrast image, thereby alleviating a sense of unnaturalness. For extracting the semantic area that is the target of precision evaluation and error (loss) calculation, known rule-based or machine-learning-based image processing can be used. Since the non-masked area in the FA examination image is a fixed area that is determined depending on the imaging apparatus 10, it may be extracted mechanically and be designated as the target of precision evaluation and error (loss) calculation.

Having been described here is a method of updating (optimizing) the parameters that constitute the network model 2521 on the basis of the error between the ground truth tensor Te122 and the output tensor Te112 outputted by the network model 2521 for the purpose of training the image generation model 2520. However, in the present embodiment, this method is a non-limiting example. The parameters that constitute the network model 2521 may be updated by applying thereto a technique related to a generative adversarial network (GAN) based on an image input such as Conditional GAN, which is known deep learning technology. For example, the parameters that constitute the network model 2521 may be updated while performing the following discrimination about the contrast effect image generated by the network model 2521 corresponding to Generator Network in Conditional GAN. Specifically, the parameters that constitute the network model 2521 may be updated while discriminating, by Discriminator Network, whether the contrast effect image is genuine one (an FA examination image) or fake one (an image that resembles an FA examination image).

The image generation model 2520 having been trained through the learning processing described above is capable of outputting a moving-picture contrast effect image that depicts a contrast effect having a plausible likelihood based on the teacher data group assigned for training among the data set, upon receiving an input of an OCTA image. That is, it is possible to output a pseudo contrast image (contrast effect image) that resembles an FA examination image in a moving-picture format depicting time-lapse changes in contrast effect, like those acquired in FA examinations.

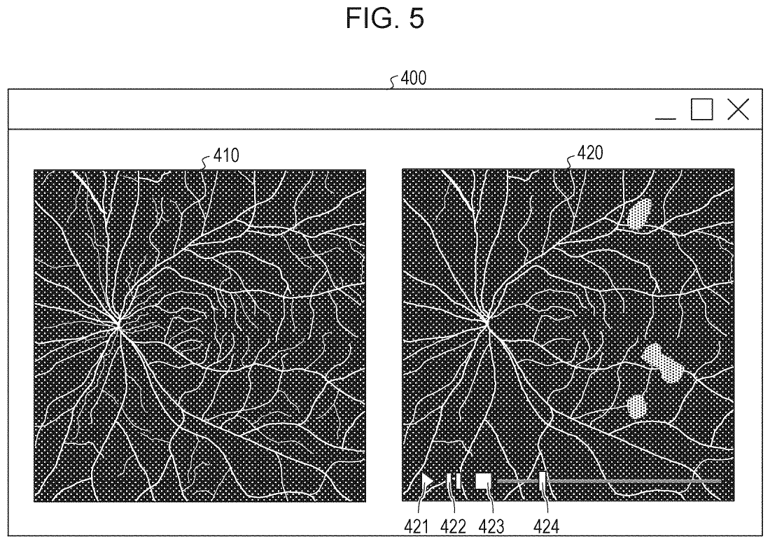

FIG. 5 is a diagram illustrating an example of a GUI screen 400 displayed on the display 230 in the image generation apparatus 20 according to the first embodiment.

The display unit 253 performs processing of displaying the GUI (Graphical User Interface) screen 400 illustrated in FIG. 5 on the display 230. Specifically, the display unit 253 performs processing of displaying the medical image acquired by the image acquisition unit 251 (in the present embodiment, the OCTA image) in an image display area 410 of the GUI screen 400 illustrated in FIG. 5. In addition, the display unit 253 performs processing of displaying the contrast effect image outputted from the outputting unit 252 in an image display area 420 of the GUI screen 400 illustrated in FIG. 5. More particularly, in the present embodiment, the display unit 253 performs processing of displaying the moving-picture contrast effect image in the image display area 420. Therefore, the operator can observe the contrast effect image by viewing the image display area 420 of the GUI screen 400. Operation tools that enable the operator to perform the movie operation of the contrast effect image are provided in the image display area 420 of the GUI screen 400. As the operation tools, a play button 421 for starting the play of the movie, a pause button 422 for pausing the play of the movie, a stop button 423 for stopping the play of the movie, and a seek bar 424 for changing the play position of the movie are provided in the image display area 420. The movie of the contrast effect image displayed in the image display area 420 may be automatically started to be played, or may be in a stopped state at a play position corresponding to contrast time moment that is useful for making a diagnosis. In the GUI screen 400 illustrated in FIG. 5, the OCTA image, which is the medical image acquired by the image acquisition unit 251, is displayed in the image display area 410 for the purpose of increasing diagnosis efficiency during observation by comparing it with the contrast effect image displayed in the image display area 420.

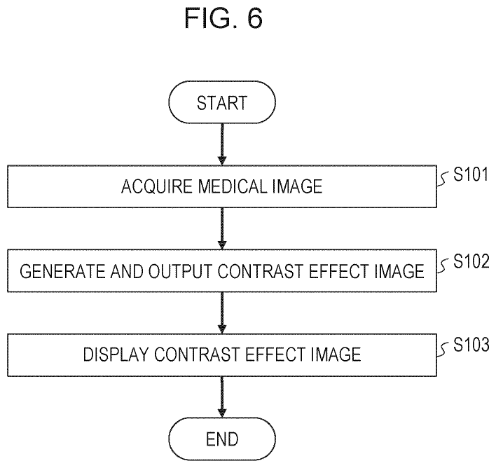

FIG. 6 is a flowchart illustrating an example of processing steps in a method of controlling the image generation apparatus 20 according to the first embodiment.

Upon the start of processing illustrated in the flowchart of FIG. 6, first, in step S101, the image acquisition unit 251 acquires an OCTA image, which is a medical image, from the imaging apparatus 10, for example.

Next, in step S102, the outputting unit 252 generates and outputs a contrast effect image that depicts a contrast effect corresponding to contrast time that includes contrast time moment of a plurality of points in time on the basis of the OCTA image acquired in step S101. Specifically, in the present embodiment, the outputting unit 252 outputs a contrast effect image that is a pseudo contrast image that resembles an FA examination image in a moving-picture format depicting time-lapse changes in contrast effect corresponding to contrast time.

Next, in step S103, the display unit 253 displays the OCTA image acquired in step S101 in the image display area 410 of the GUI screen 400 illustrated in FIG. 5 and displays the moving-picture contrast effect image outputted in step S102 in the image display area 420 thereof.

Upon the end of processing in step S103, the processing illustrated in the flowchart of FIG. 6 ends.

As explained above, in the image generation apparatus 20 according to the first embodiment, the image acquisition unit 251 acquires an OCTA image, which is a medical image, from the imaging apparatus 10, for example. Then, the outputting unit 252 outputs a contrast effect image that depicts a contrast effect corresponding to contrast time that includes contrast time moment of a plurality of points in time (a contrast effect image in a moving-picture format depicting a contrast effect) on the basis of the OCTA image acquired by the image acquisition unit 251. For example, in a case where the contrast time comprises contrast time moment of a plurality of points in time in a time-lapse manner, a contrast effect image in a moving-picture format depicting time-lapse changes in contrast effect is outputted.

With this configuration, it is possible to desirably acquire an image that depicts a contrast effect corresponding to contrast time that includes contrast time moment of a plurality of points in time. This makes it possible to desirably acquire an FA-examination-image-like image that depicts a contrast effect corresponding to the contrast time that includes the contrast time moment at which the operator wants to make an observation, thereby assisting the operator in making a decision in a diagnosis.

### First Variation Example of First Embodiment

Next, as a variation example of the first embodiment described above, a first variation example of the first embodiment will now be described.

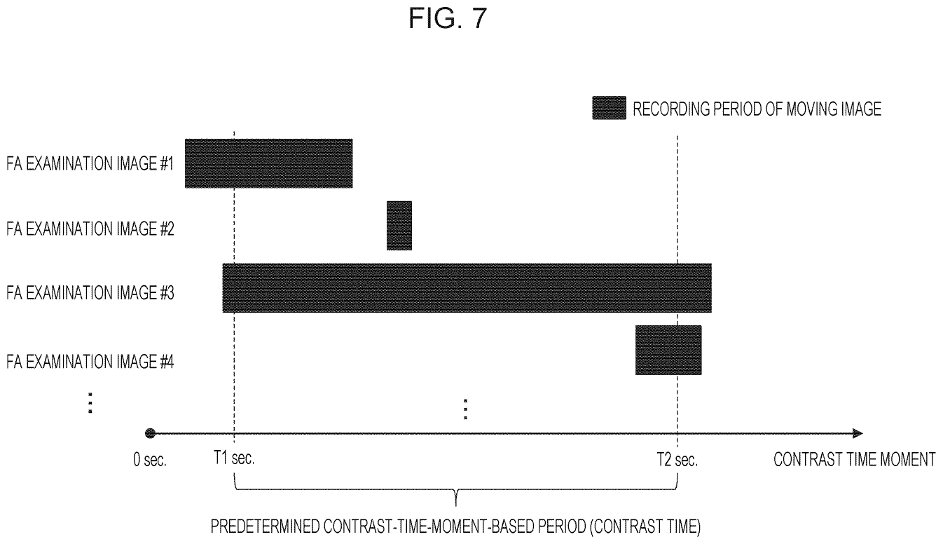

FIG. 7 is a diagram illustrating the first variation example of the first embodiment for explaining a “contrast-time-moment-based period” (contrast time) during which an FA examination image(s) is recorded, wherein the FA examination image is a moving image included in teacher data that is used when the image generation model 2520 is trained.

The FA examination image, which is a moving image included in teacher data that is used when the image generation model 2520 is trained, may be, as illustrated in FIG. 7, an FA examination image of only a part of a predetermined contrast-time-moment-based period (contrast time) from time moment of T1 sec. to time moment of T2 sec. The predetermined contrast-time-moment-based period (contrast time) may preferably be covered when the recording periods of all of the FA examination images are merged. In a case where a contrast-time-moment-based period (contrast time) for which there is a clinical need of observation can be specified or where a contrast-time-moment-based period (contrast time) that is of the operator's particular interest of observation can be specified, FA examination images that cover this contrast-time-moment-based period (contrast time) may preferably be put into the teacher data group in a focused manner. That is, the FA examination image group (contrast image group) included in the training data may preferably include more FA examination images captured in the contrast time that includes the contrast time moment at which the operator wants to make an observation than FA examination images captured in contrast time that includes other contrast time moment. This improves the image generation precision (the likelihood of depicting by the contrast effect image) of the image generation model 2520 for this contrast-time-moment-based period (contrast time) and is therefore effective. In this case, only the contrast time moment corresponding to the play position(s) of the moving-picture frame image(s) that exists is used for precision evaluation and error (loss) calculation.

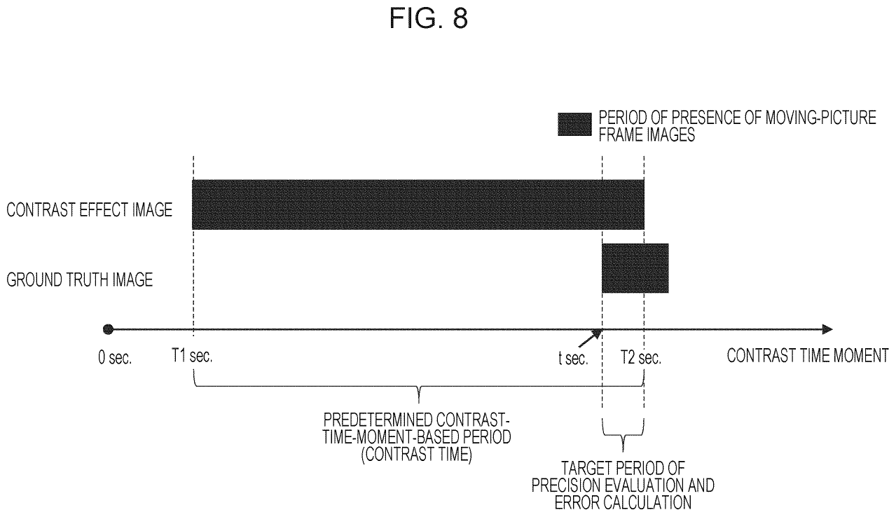

FIG. 8 is a diagram illustrating the first variation example of the first embodiment, and illustrating an example of a relationship between the contrast effect image that is a moving image outputted by the image generation model 2520 and the ground truth image (FA examination image) that is a moving image included in the teacher data.

For example, when precision evaluation or error (loss) calculation is performed on the contrast effect image and the ground truth image (FA examination image) that are illustrated in FIG. 8, a period from contrast time moment of t sec. to contrast time moment of T2 sec., which is the contrast-time-moment-based period (contrast time) of the moving-picture frame image group existing in the ground truth image, is taken as the target period for the calculation.

In the first variation example of the first embodiment, consideration is given also to a case where the FA examination image that is a moving image included in the teacher data is not recorded in such a way as to cover the predetermined contrast-time-moment-based period (contrast time). With the first variation example of the first embodiment, even in such a case, it is possible to desirably acquire a pseudo image (contrast effect image) that resembles an FA examination image in a moving-picture format depicting time-lapse changes in contrast effect on the basis of an OCTA image. This makes it possible to desirably acquire an FA-examination-image-like image that depicts a contrast effect corresponding to the contrast time that includes the contrast time moment at which the operator wants to make an observation, thereby assisting the operator in making a decision in a diagnosis.

### Second Variation Example of First Embodiment

Next, as another variation example of the first embodiment described above, a second variation example of the first embodiment will now be described.

In the first embodiment described above, FA examination images that have different imaging-range sizes (i.e., angles of field) may exist in a mixed manner as the FA examination images in the teacher data group that is used when the image generation model 2520 is trained. In this regard, it is sometimes difficult to perform anatomical alignment if there is a wide difference in imaging-range size between an OCTA image and an FA examination image. For example, if the imaging range of the OCTA image and the imaging range of the FA examination image are almost the same as each other, the common regions and blood vessels of the target of examination (in the present embodiment, the subject eye) are depicted in both of these images, which makes it easier to perform anatomical alignment properly.

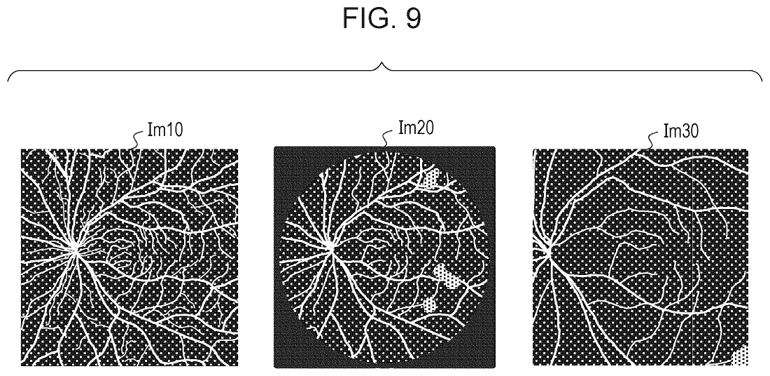

FIG. 9 is a diagram illustrating the second variation example of the first embodiment, and illustrating an example of an OCTA image and an FA examination image. In FIG. 9, a wide-area OCTA image Im10 capturing a wide area, a wide-area FA examination image Im20 capturing a wide area, and a narrow-area FA examination image Im30 capturing a narrow area are illustrated. When it is attempted to anatomically align the wide-area OCTA image Im10 illustrated in FIG. 9 with the narrow-area FA examination image Im30 illustrated therein, this anatomical alignment is sometimes difficult because there is a wide difference between these two images as to how the region and blood vessels are depicted, in conjunction with a difficulty arising from a fundamental fact that these two images have been captured respectively by imaging apparatuses that are different from each other. In such a case, it is possible to improve the result of the anatomical alignment by using the wide-area FA examination image Im20, which is an image acquired by taking a shot of a wider area of the same target of examination.

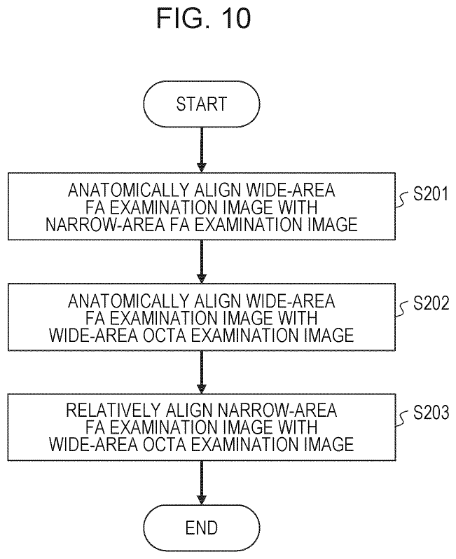

FIG. 10 is a flowchart illustrating the second variation example of the first embodiment, and illustrating an example of processing steps in processing for alignment of an OCTA image and an FA examination image.

Upon the start of processing illustrated in the flowchart of FIG. 10, first, in step S201, the image generation model 2520 anatomically aligns the wide-area FA examination image Im20 illustrated in FIG. 9 with the narrow-area FA examination image Im30 illustrated therein. At this time, the anatomical alignment is feasible because both images have been acquired from the imaging apparatus 10 that is the same one.

Next, in step S202, the image generation model 2520 anatomically aligns the wide-area FA examination image Im20 with the wide-area OCTA image Im10. At this time, the anatomical alignment is feasible because both images have been acquired through wide-area capturing.

Next, in step S203, the image generation model 2520 performs relative alignment of the wide-area OCTA image Im10 and the narrow-area FA examination image Im30. Specifically, the image generation model 2520 performs the alignment in step S203 by combining information on deformation at the time of performing the anatomical alignment in step S201 with information on deformation at the time of performing the anatomical alignment in step S202.

With the second variation example of the first embodiment, even in a case where there is a wide difference in imaging-range size between an OCTA image and an FA examination image, it is possible to perform better anatomical alignment. Consequently, it is possible to bring the manner of depicting the contrast effect by the contrast effect image outputted by the image generation model 2520 closer to a real FA examination image. That is, it is possible to desirably acquire a pseudo image (contrast effect image) that resembles an FA examination image in a moving-picture format depicting time-lapse changes in contrast effect on the basis of an OCTA image. This makes it possible to desirably acquire an FA-examination-image-like image that depicts a contrast effect corresponding to the contrast time that includes the contrast time moment at which the operator wants to make an observation, thereby assisting the operator in making a decision in a diagnosis. Third Variation Example of First Embodiment

Next, as another variation example of the first embodiment described above, a third variation example of the first embodiment will now be described.

With regard to the data set for training the image generation model 2520 according to the first embodiment described above, the OCTA images (medical image group) that constitute the data set may be replaced with images of any other kind that record a state of the fundus of the subject eye.

For example, as the image of any other kind, three-dimensional motion contrast data acquired by an OCT apparatus, a two-dimensional OCT image, or a three-dimensional OCT image may be used. For example, as the image of any other kind, a fundus image acquired by a fundus camera or a scanning laser ophthalmoscope (SLO) image acquired by a scanning laser ophthalmoscope may be used.

For example, a mixture of an OCTA image and the image of any other kind mentioned above may be used. Specifically, for example, a fundus image that is a 3-channel RGB color image may be mixed with an OCTA image that is a 1-channel grayscale image on a channel axis to obtain a 4-channel image. When this is performed, it is preferable if the anatomical position of the fundus image and the anatomical position of the OCTA image match; therefore, anatomical alignment is performed. Alternatively, if the imaging apparatus 10 has both a function of a fundus camera and a function of an OCT apparatus, the anatomical position of the acquired fundus image and the anatomical position of the acquired OCTA image could already match, and, if so, anatomical alignment is not needed.

In a case where an OCTA image is replaced with an image of any other kind mentioned above, “OCTA image” described above in the first embodiment should read as “image of any other kind” described above. Based on the above, it is possible to desirably acquire a pseudo image (contrast effect image) that resembles an FA examination image in a moving-picture format depicting time-lapse changes in contrast effect on the basis of “image of any other kind” described above. This makes it possible to desirably acquire an FA-examination-image-like image that depicts a contrast effect corresponding to the contrast time that includes the contrast time moment at which the operator wants to make an observation, thereby assisting the operator in making a decision in a diagnosis.

### Second Embodiment

Next, a second embodiment will now be described. In the second embodiment described below, description of matters that are the same as those having been described in the first embodiment above will be omitted, and matters that are different from those having been described in the first embodiment above will be described.

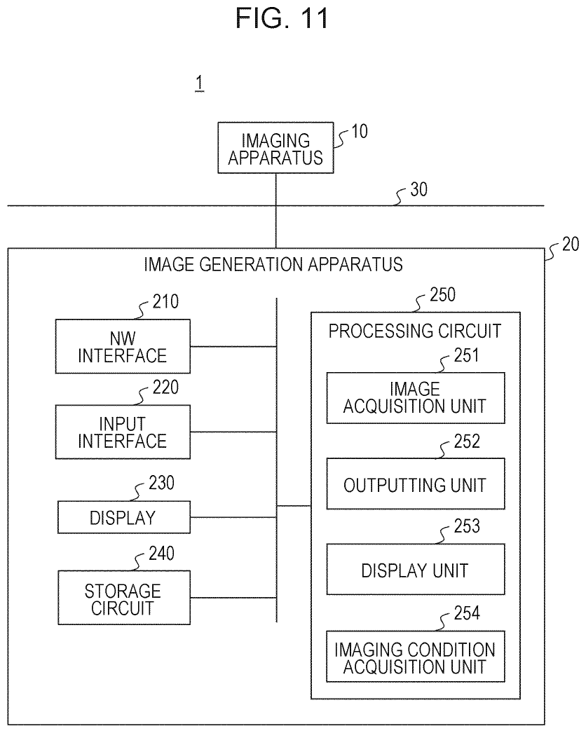

FIG. 11 is a diagram illustrating an example of a schematic configuration of the image generation system 1 including the image generation apparatus 20 according to the second embodiment. In FIG. 11, the same reference signs are assigned to components, etc. that are the same as those illustrated in FIG. 1, and a detailed explanation thereof is omitted.

Compared with the configuration of the image generation apparatus 20 according to the first embodiment illustrated in FIG. 1, the configuration of the image generation apparatus 20 according to the second embodiment illustrated in FIG. 11 additionally includes an imaging condition acquisition unit 254 in the processing circuit 250.

The imaging condition acquisition unit 254 has a function of acquiring an imaging condition(s) that includes contrast time that includes contrast time moment of at least one point in time.

First, the outputting unit 252 generates a “for-extraction-use” contrast effect image that is a moving image that depicts a contrast effect corresponding to contrast time that includes contrast time moment of a plurality of points in time on the basis of a medical image that is a still image acquired by the image acquisition unit 251, similarly to the first embodiment. Then, the outputting unit 252 extracts, from the moving-picture frame image group that constitutes the for-extraction-use contrast effect image, the moving-picture frame image corresponding to the contrast time included in the imaging condition acquired by the imaging condition acquisition unit 254, and outputs the extraction result as a final contrast effect image. Specifically, the contrast effect image according to the present embodiment is a pseudo contrast image that resembles an FA examination image in a still-picture format depicting a contrast effect corresponding to the contrast time moment of the designated point in time, like those acquired in FA examinations. For easier understanding, it is assumed here that the imaging condition acquisition unit 254 according to the present embodiment acquires information on contrast time moment only as the imaging condition.

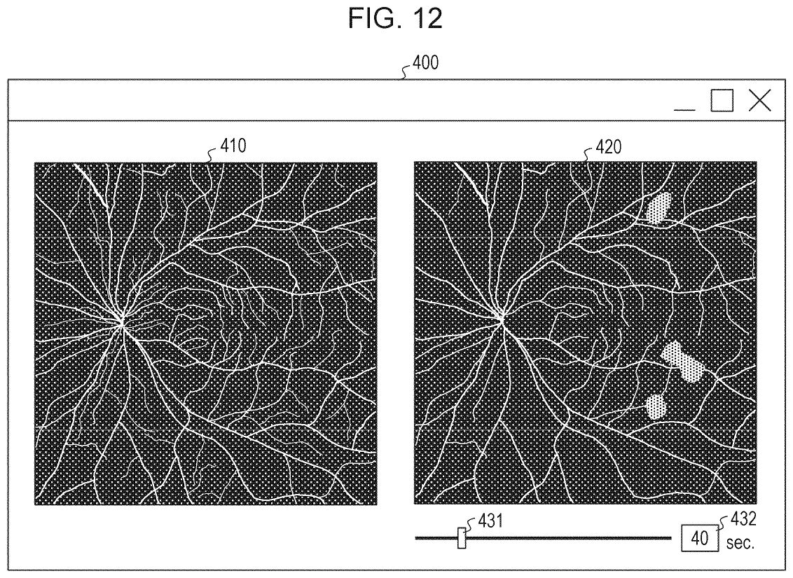

FIG. 12 is a diagram illustrating an example of the GUI screen 400 displayed on the display 230 in the image generation apparatus 20 according to the second embodiment. In FIG. 12, the same reference signs are assigned to components, etc. that are the same as those illustrated in FIG. 5, and a detailed explanation thereof is omitted.

Compared with the configuration of the GUI screen 400 according to the first embodiment illustrated in FIG. 5, mainly, the configuration of the GUI screen 400 according to the second embodiment illustrated in FIG. 12 additionally includes a contrast time moment designation slider 431 and a contrast time moment designation text box 432.

The contrast time moment set as the imaging condition can be designated by, for example, operating the contrast time moment designation slider 431 or the contrast time moment designation text box 432 illustrated in FIG. 12 by the operator using the input interface 220. For example, FIG. 12 illustrates an exemplary case where the time moment of “40 sec.” after the reference point in time is designated as the contrast time moment. The method of designating the contrast time moment is not limited to the one described here. It may be replaced with any other method by means of which the same object can be achieved. Though the GUI screen 400 that allows the operator to designate contrast time moment has been described here, contrast time moment that is preset in the image generation system 1 according to the second embodiment may be inputted.

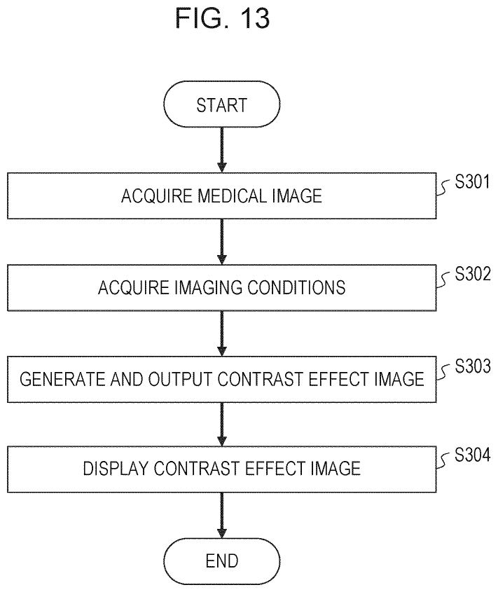

FIG. 13 is a flowchart illustrating an example of processing steps in a method of controlling the image generation apparatus 20 according to the second embodiment.

Upon the start of processing illustrated in the flowchart of FIG. 13, first, in step S301, the image acquisition unit 251 acquires an OCTA image, which is a medical image, from the imaging apparatus 10, for example.

Next, in step S302, the imaging condition acquisition unit 254 acquires an imaging condition that includes contrast time that includes contrast time moment of at least one point in time. Specifically, in the present embodiment, the contrast time moment is acquired as the imaging condition.

Next, in step S303, the outputting unit 252 generates and outputs a contrast effect image that depicts a contrast effect corresponding to the contrast time moment on the basis of the OCTA image acquired in step S301 and on the basis of the imaging condition (contrast time moment) acquired in step S302. Specifically, in the present embodiment, the outputting unit 252 outputs a contrast effect image that is a pseudo contrast image that resembles an FA examination image in a still-picture format depicting a contrast effect corresponding to the contrast time moment.

Next, in step S304, the display unit 253 displays the OCTA image acquired in step S301 in the image display area 410 of the GUI screen 400 illustrated in FIG. 12 and displays the contrast effect image outputted in step S303 in the image display area 420 thereof.

Upon the end of processing in step S304, the processing illustrated in the flowchart of FIG. 13 ends.

As explained above, in the image generation apparatus 20 according to the second embodiment, the image acquisition unit 251 acquires an OCTA image, which is a medical image, from the imaging apparatus 10, for example. The imaging condition acquisition unit 254 acquires an imaging condition that includes contrast time that includes contrast time moment of at least one point in time. Then, the outputting unit 252 outputs a contrast effect image that depicts a contrast effect corresponding to the contrast time on the basis of the OCTA image acquired by the image acquisition unit 251 and on the basis of the imaging condition acquired by the imaging condition acquisition unit 254.

With this configuration, it is possible to desirably acquire an image that depicts a contrast effect corresponding to contrast time (in the present embodiment, contrast time moment) that includes contrast time moment of a certain point in time. More specifically, the image generation apparatus 20 according to the second embodiment is capable of desirably acquiring an FA-examination-image-like image that depicts a contrast effect corresponding to the contrast time moment at which the operator wants to make an observation, thereby assisting the operator in making a decision in a diagnosis.

### Third Embodiment

Next, a third embodiment will now be described. In the third embodiment described below, description of matters that are the same as those having been described in the first and second embodiments above will be omitted, and matters that are different from those having been described in the first and second embodiments above will be described.

The schematic configuration of an image generation system that includes an image generation apparatus according to the third embodiment is the same as the schematic configuration of the image generation system 1 that includes the image generation apparatus 20 according to the second embodiment illustrated in FIG. 11.

The outputting unit 252 according to the third embodiment outputs, on the basis of a medical image that is a still image acquired by the image acquisition unit 251, a contrast effect image that is a still image that depicts a contrast effect corresponding to the contrast time moment included in the imaging condition acquired by the imaging condition acquisition unit 254.

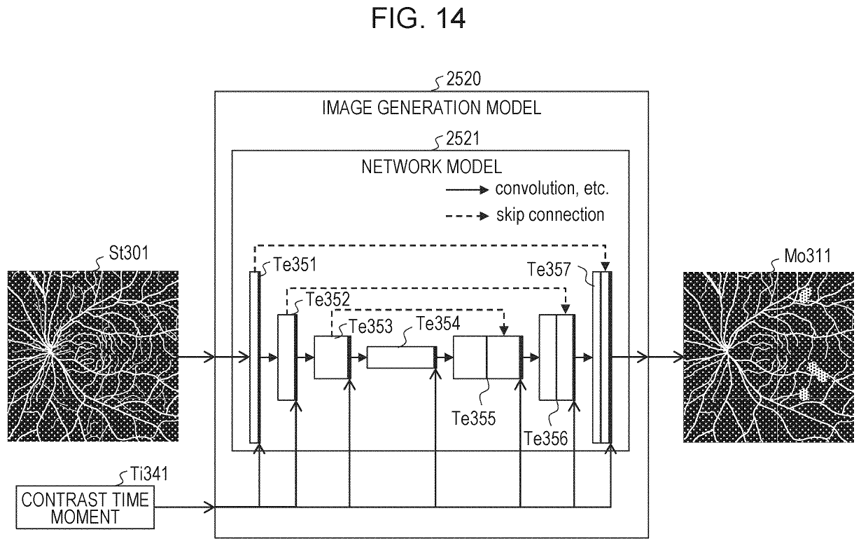

FIG. 14 is a diagram for explaining the concept of the image generation model 2520 of the outputting unit 252 in the image generation apparatus 20 according to the third embodiment. In FIG. 14, the same reference signs are assigned to components, etc. that are the same as those illustrated in FIG. 2, and a detailed explanation thereof is omitted.

The outputting unit 252 according to the third embodiment includes the image generation model 2520 illustrated in FIG. 14. The image generation model 2520 illustrated in FIG. 14 includes the U-Net-based network model 2521 as the image processing system based on deep learning technology. Though U-Net is taken as an example in the present embodiment, any other network model with which the same object can be achieved may be adopted.

The image generation model 2520 illustrated in FIG. 14 receives an input image St301, which is a still-picture medical image, and contrast time moment Ti341 as inputs, and generates a still-picture contrast effect image that depicts a contrast effect corresponding to the contrast time moment Ti341 on the basis of the input image St301. Specifically, the image generation model 2520 illustrated in FIG. 14 inputs, into the network model 2521, the still-picture input image St301 after transforming it into a tensor and the contrast time moment Ti341 after transforming it into a tensor. Then, the image generation model 2520 illustrated in FIG. 14 applies still-picture transformation to a tensor outputted from the network model 2521 and outputs this still image as an output image Mo311.

In a case where U-Net is adopted as the network model 2521, there is a need to modify the U-Net. Specifically, a scalar value T that represents the contrast time moment Ti341 is given to at least one tensor space axis among the number of channels, height, and width of at least one of tensors generated in the intermediate layer of the network model 2521. “Tensors generated in the intermediate layer” mentioned here correspond to tensors Te351 to Te357 in FIG. 14. Though the scalar value T is given to every tensor Te351 to Te357 in FIG. 14, for example, a configuration of giving the scalar value T to the tensor Te351 only, a configuration of giving the scalar value T to the tensors Te355 to Te357, and the like are possible.

The scalar value T is a scalar value determined on the basis of the contrast time moment Ti341, for example, through division of the contrast time moment Ti341 in unit of millisecond by a constant, etc. As a specific method of the giving, for example, let us consider a case where the original shape of a tensor before the scalar value T is given thereto is “B×C×H×W”, where B denotes mini-batch size, C denotes the number of channels, H denotes height, and W denotes width. In the case of this shape, the number of channels is extended into a shape of “B×(C+1)×H×W”, and processing of filling the value of the extended tensor region with the scalar value T is added, and, in addition, the structure of the network model 2521 is altered so as to make it possible to process the extended tensor. Alternatively, if the number of channels is two or more, the value of an arbitrary tensor region corresponding to one channel may be filled with the scalar value T, instead of the tensor extension. For the purpose of increasing the image generation precision of the image generation model 2520 (the likelihood of the output image Mo311) or increasing computational efficiency, sometimes the network model 2521 that deals with normalized input and output tensors is used. For the range of the value of the tensor generated by the network model 2521 (e.g., a range from −10.0 to 10.0), it is conceivable that a relatively large value such as, for example, 40000, representing 40000 milliseconds, will be set as the scalar value T that represents the contrast time moment Ti341. In this case, since there is a possibility that a model with low image generation precision might be learned, the scalar value T may be normalized; for example, it may be converted into a value from 0 to 1 by division by the maximum value that can be inputted into the image generation model 2520.

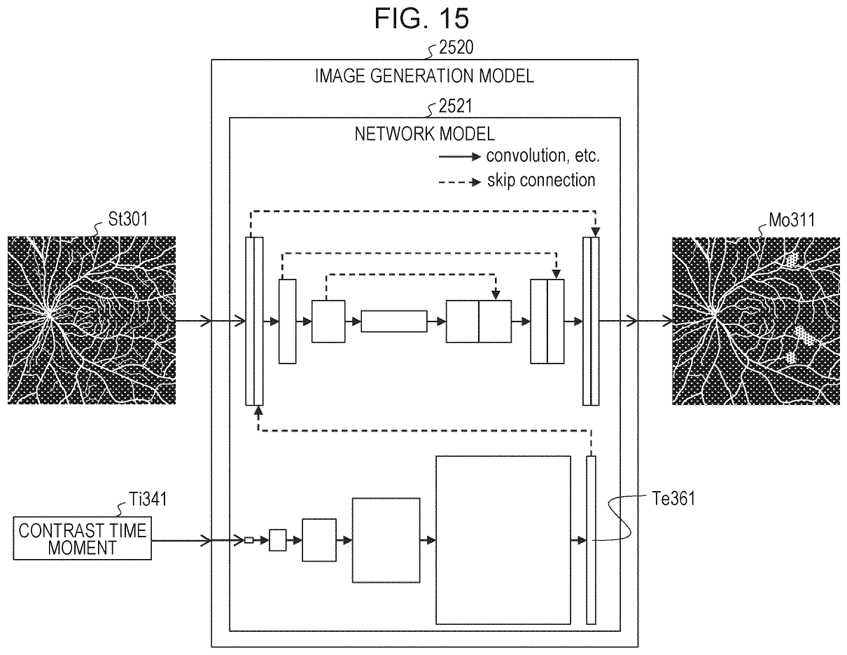

The object of applying the above manipulation to the tensors, which has been described with reference to FIG. 14, is to cause the network model 2521 to process the input image St301, which is an OCTA image, and the contrast time moment Ti341 by inputting information about contrast time moment into the image generation model 2520. Therefore, in the present embodiment, the method is not limited to the one having been described with reference to FIG. 14. With reference to FIG. 15, an example of another method will now be described.

FIG. 15 is a diagram for explaining the concept of the image generation model 2520 of the outputting unit 252 in the image generation apparatus 20 according to the third embodiment. In FIG. 15, the same reference signs are assigned to components, etc. that are the same as those illustrated in FIGS. 2 and 14, and a detailed explanation thereof is omitted.

For example, as another method, as illustrated in FIG. 15, a method of configuring the network model 2521 by combining non-modified U-Net with a known decoder network can also be used. Specifically, first, the scalar value T that represents the contrast time moment Ti341 is inputted into the decoder network. Then, an up-sampled tensor Te361 outputted from the decoder network is concatenated with the tensor of the OCTA image inputted into the U-Net, and the U-Net outputs a tensor of the contrast effect image. The configuration illustrated in FIG. 15 also makes it possible to acquire the contrast effect image as the output image Mo311 from the image generation model 2520 by causing the network model 2521 to process the input image St301, which is the OCTA image, and the contrast time moment Ti341.

Applying the above manipulation to the tensors makes it possible to cause the image generation model 2520 to output a contrast effect image that is a still image that depicts a contrast effect corresponding to arbitrary contrast time moment by inputting information on the contrast time moment Ti341 into the network model 2521. The method of inputting information on the contrast time moment Ti341 into the network model 2521 is not limited to the method described in the present embodiment. Any other method with which the same object can be achieved may be used. For example, a method of manipulating the pixel values of the input image St301 by means of a value related to the contrast time moment Ti341, or a method of adding a new image channel to the input image St301 and setting pixel values related to the contrast time moment Ti341, can also be used. Furthermore, a method of additionally inputting an image generated on the basis of the contrast time moment Ti341 into the network model 2521 can also be used.

A data set for training the image generation model 2520, which includes the above-described U-Net-based network model 2521, will now be described. A data set has a structure of a teacher data group acquired from a plurality of examination targets, wherein an OCTA image that is a still image acquired by taking a shot of the same examination target, an FA examination image captured at certain contrast time moment, and the contrast time moment of the FA examination image are “paired” to constitute each one piece of teacher data in the group. The examination target is, in the present embodiment, the subject eye. For one OCTA image, a plurality of FA examination images (contrast image group) acquired by taking time-lapse shots, and the contrast time moment group (imaging condition group) corresponding to the FA examination image group, may exist.

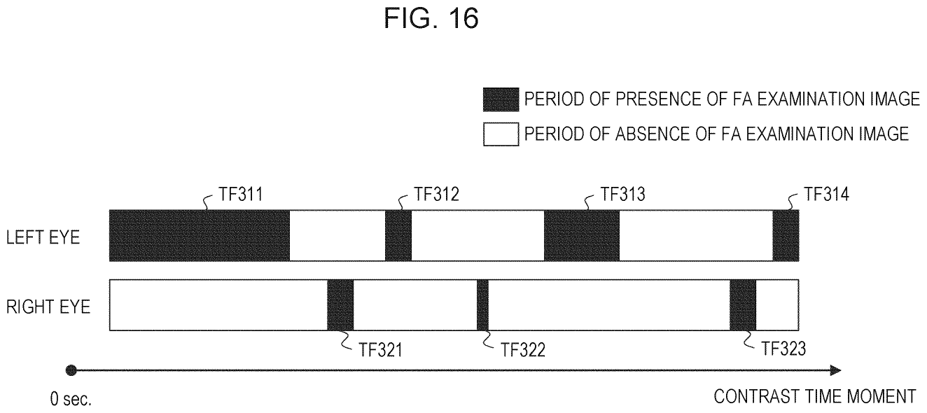

FIG. 16 is a diagram illustrating the third embodiment, and illustrating an example of periods of presence/absence of left-eye/right-eye FA examination images included in teacher data that is used when the image generation model 2520 is trained. In an FA examination, the left eye and the right eye are subjected to imaging alternately after a contrast medium is administered; therefore, for example, time slots of FA examination image presence could be in a distribution illustrated in FIG. 16. Among these time slots, in a long time slot such as a time slot TF311, a moving image could be captured as an FA examination image. In the present embodiment, in a case where a moving image is acquired, moving-picture frame images that constitute the moving image may be extracted as a still image group, a contrast time moment group corresponding to the moving-picture frame images may be identified, and each of them may be paired with the corresponding OCTA image of the subject eye, to be used as teacher data.

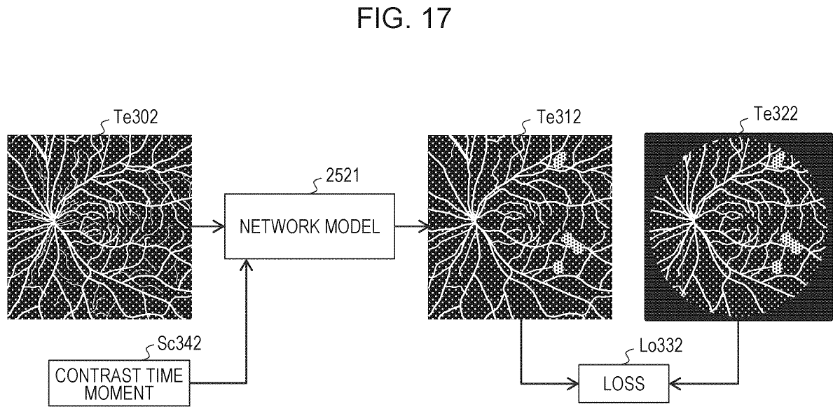

FIG. 17 is a diagram for explaining the training of the image generation model 2520 of the outputting unit 252 in the image generation apparatus 20 according to the third embodiment. In FIG. 17, the same reference signs are assigned to components, etc. that are the same as those illustrated in FIGS. 14 and 15, and a detailed explanation thereof is omitted. With reference to FIG. 17, the training of the image generation model 2520 using certain one pair of teacher data, that is, processing for updating the parameters that constitute the network model 2521 included in the image generation model 2520, will now be described.

First, in FIG. 17, an input tensor Te302, which is a tensor transformed from the OCTA image included in the teacher data, and a scalar value Sc342, which represents the contrast time moment Ti341 included in the same teacher data, are inputted into the network model 2521. Upon the input, an output tensor Te312, which corresponds to the contrast effect image that is a still image, is outputted from the network model 2521. Next, the image generation model 2520 calculates a loss Lo332, which is an error of the output tensor Te312 compared with a ground truth tensor Te322, which is a tensor transformed from the FA examination image that is a still image captured at the contrast time moment Ti341 and included in the same teacher data. Finally, the image generation model 2520 updates the parameters that constitute the network model 2521 in such a way as to make the loss Lo332 small. This series of update processing is repeated while using a teacher data group assigned for training among the data set until the network model 2521 becomes trained enough.

The image generation model 2520 having been trained through the learning processing described above is capable of outputting a still-picture contrast effect image that depicts a contrast effect having a plausible likelihood based on the teacher data group assigned for training among the data set, upon receiving an input of an OCTA image. That is, it is possible to output a pseudo image (contrast effect image) that resembles an FA examination image in a still-picture format depicting a contrast effect corresponding to the designated contrast time moment, like those acquired in FA examinations.

Processing steps in a method of controlling the image generation apparatus 20 according to the third embodiment are the same as the processing steps illustrated in the flowchart of FIG. 13, which relates to the method of controlling the image generation apparatus 20 according to the second embodiment. With reference to the flowchart of FIG. 13, the processing steps in the method of controlling the image generation apparatus 20 according to the third embodiment will now be described.

In the third embodiment, upon the start of processing illustrated in the flowchart of FIG. 13, first, in step S301, the image acquisition unit 251 acquires an OCTA image, which is a medical image, from the imaging apparatus 10, for example.

Next, in step S302, the imaging condition acquisition unit 254 acquires an imaging condition that includes contrast time that includes contrast time moment of at least one point in time. Specifically, in the present embodiment, the contrast time moment is acquired as the imaging condition.

Next, in step S303, the outputting unit 252 generates and outputs a contrast effect image that depicts a contrast effect corresponding to the contrast time moment on the basis of the OCTA image acquired in step S301 and on the basis of the imaging condition (contrast time moment) acquired in step S302. Specifically, in the present embodiment, the outputting unit 252 outputs a contrast effect image that is a pseudo contrast image that resembles an FA examination image in a still-picture format depicting a contrast effect corresponding to the contrast time moment.

Next, in step S304, the display unit 253 displays the OCTA image acquired in step S301 in the image display area 410 of the GUI screen 400 illustrated in FIG. 12 and displays the contrast effect image outputted in step S303 in the image display area 420 thereof.

Upon the end of processing in step S304, the processing illustrated in the flowchart of FIG. 13 ends.

As explained above, in the image generation apparatus 20 according to the third embodiment, the image acquisition unit 251 acquires an OCTA image, which is a medical image, from the imaging apparatus 10, for example. The imaging condition acquisition unit 254 acquires an imaging condition that includes contrast time that includes contrast time moment of at least one point in time. Then, the outputting unit 252 outputs a contrast effect image that depicts a contrast effect corresponding to the contrast time on the basis of the OCTA image acquired by the image acquisition unit 251 and on the basis of the imaging condition acquired by the imaging condition acquisition unit 254.

With this configuration, it is possible to desirably acquire an image that depicts a contrast effect corresponding to contrast time (in the present embodiment, contrast time moment) that includes contrast time moment of a certain point in time. More specifically, the image generation apparatus 20 according to the third embodiment is capable of desirably acquiring an FA-examination-image-like image that depicts a contrast effect corresponding to the contrast time moment at which the operator wants to make an observation, thereby assisting the operator in making a decision in a diagnosis.

Moreover, compared with the image generation apparatus 20 according to the first embodiment, the image generation apparatus 20 according to the third embodiment does not output a moving image and is thus lower in terms of time cost and computation cost incurred by the outputting unit 252 and is thus more useful in an environment on which performance limitations are imposed. Furthermore, teacher data that is a moving image satisfying a predetermined contrast-time-moment-based period (contrast time) is not required for the training of the image generation model 2520 of the outputting unit 252. That is, it does not matter even if the FA examination images included in pieces of teacher data correspond to different points of contrast time moment. This makes it easy to gather pieces of teacher data and thus makes it possible to increase the possibility of depicting a contrast effect that more closely resembles a real contrast image.

### First Variation Example of Third Embodiment

Next, as a variation example of the third embodiment described above, a first variation example of the third embodiment will now be described.

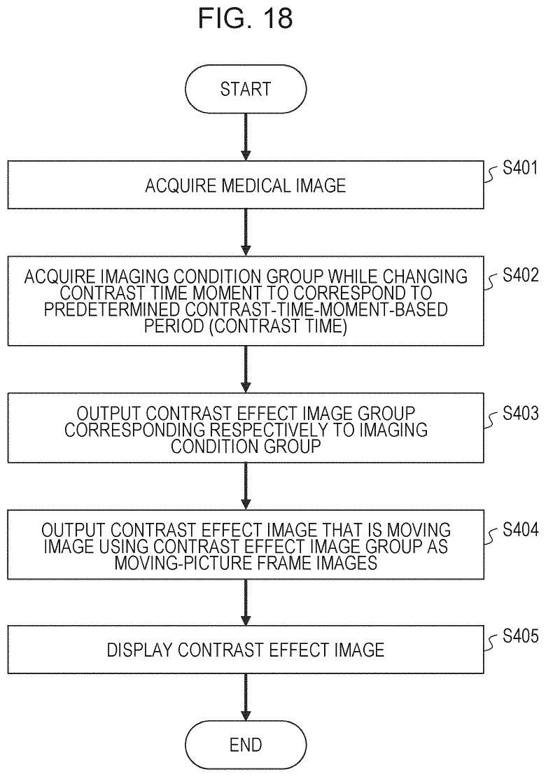

FIG. 18 is a flowchart illustrating an example of processing steps in a method of controlling the image generation apparatus 20 according to the first variation example of the third embodiment. Through processing illustrated in the flowchart of FIG. 18, a contrast effect image in a moving-picture format can also be outputted.

Upon the start of processing illustrated in the flowchart of FIG. 18, first, in step S401, the image acquisition unit 251 acquires a medical image from the imaging apparatus 10, for example. In the first variation example of the third embodiment, an OCTA image is acquired as the medical image.

Next, in step S402, the imaging condition acquisition unit 254 acquires an imaging condition group while changing contrast time moment in such a way as to correspond to a predetermined contrast-time-moment-based period (contrast time). For example, suppose that the operator wants to observe a contrast effect at one-second intervals with the predetermined contrast-time-moment-based period designated as “from 0 sec. to 200 sec.”; in this case, a group comprised of two hundred one imaging conditions (contrast time moment) that are generated while changing the contrast time moment to 1, 2, . . . , 200 sec. is acquired.

Next, in step S403, the outputting unit 252 outputs a contrast effect image group corresponding respectively to the imaging condition group (contrast time moment group) acquired in step S402, on the basis of the OCTA image acquired in step S401. Specifically, in step S403, the group of contrast effect images each of which is a pseudo contrast image that resembles an FA examination image in a still-picture format depicting a contrast effect corresponding to each in the contrast time moment group is outputted.

Next, in step S404, the outputting unit 252 outputs a contrast effect image that is a moving image using the contrast effect image group outputted in step S403 as moving-picture frame images.

Next, in step S405, the display unit 253 displays the OCTA image acquired in step S401 in the image display area 410 of the GUI screen 400 illustrated in FIG. 5 and displays the moving-picture contrast effect image outputted in step S404 in the image display area 420 thereof.

Upon the end of processing in step S405, the processing illustrated in the flowchart of FIG. 18 ends.

With the first variation example of the third embodiment, it is possible to desirably acquire a pseudo image (contrast effect image) that resembles an FA examination image in a moving-picture format depicting time-lapse changes in contrast effect on the basis of an OCTA image. This makes it possible to desirably acquire an FA-examination-image-like image that depicts a contrast effect corresponding to the contrast time that includes the contrast time moment at which the operator wants to make an observation, thereby assisting the operator in making a decision in a diagnosis.

Second Variation Example of Third Embodiment Next, as another variation example of the third embodiment described above, a second variation example of the third embodiment will now be described.

With regard to the data set for training the image generation model 2520 according to the third embodiment described above, the FA examination images that constitute the data set may be replaced with images of any other kind from which it is possible to know the state of the contrast effect in the target of examination.

For example, as an image of any other kind, an area demarcation image that illustrates a range of contrast medium leakage known from the FA examination image acquired at certain contrast time moment, a contour image of the range of the leakage, or an image coloring the FA examination image by means of a color lookup table may be used.

With the second variation example of the third embodiment, it is possible to acquire the above-described image of any other kind as a contrast effect image that depicts a contrast effect corresponding to contrast time moment on the basis of an OCTA image. This makes it possible to desirably acquire an image from which the state of a contrast effect corresponding to the contrast time moment at which the operator wants to make an observation can be known, thereby assisting the operator in making a decision in a diagnosis. Third Variation Example of Third Embodiment

Next, as another variation example of the third embodiment described above, a third variation example of the third embodiment will now be described.

With regard to the data set for training the image generation model 2520 according to the third embodiment, in the FA examination images that constitute the data set, an interpolation FA examination image(s) generated by interpolating a plurality of FA examination images acquired by taking shots of the same examination target in a time-lapse manner may be adopted. More particularly, as illustrated in FIG. 16, in an FA examination, there exists a “period of FA examination image absence”, which is a period during which an FA examination image is not acquired. By generating and adopting an image corresponding to an FA examination image in the “period of FA examination image absence” through interpolation processing, it is possible to improve the image generation precision (the likelihood of depicting by the contrast effect image) of the image generation model 2520.

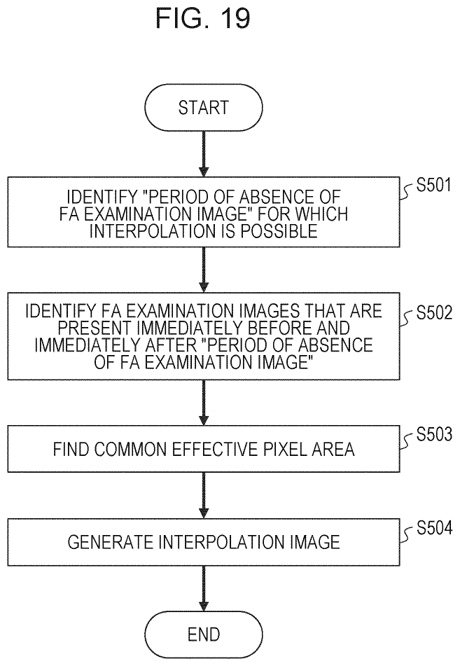

FIG. 19 is a flowchart illustrating the third variation example of the third embodiment, and illustrating an example of processing steps in interpolation image generation processing.

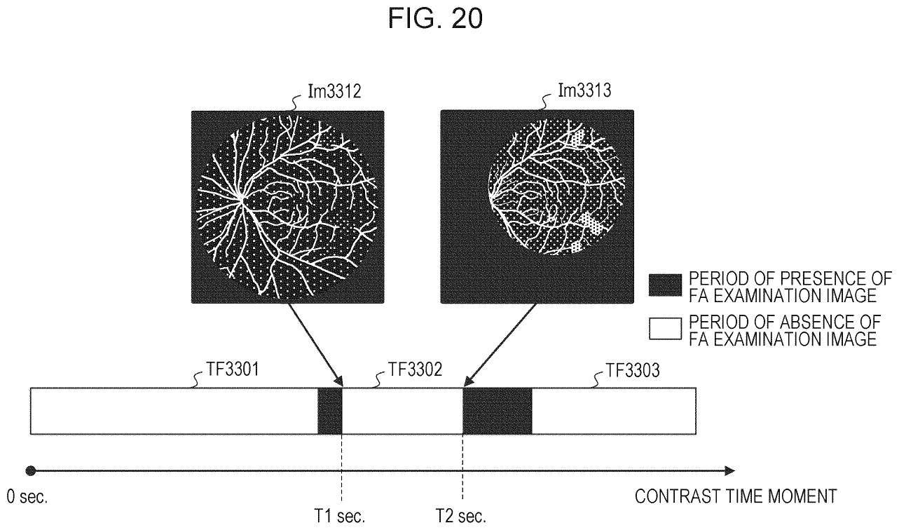

Upon the start of processing illustrated in the flowchart of FIG. 19, first, in step S501, the image generation model 2520 identifies a “period of FA examination image absence” for which interpolation is possible. The “period of FA examination image absence” for which interpolation is possible is a period immediately before which an FA examination image is present and immediately after which an FA examination image is present. FIG. 20 is a diagram illustrating the third variation example of the third embodiment, and illustrating an example of a period of presence of an FA examination image included in teacher data that is used when the image generation model 2520 is trained and a period of absence thereof. In FIG. 20, the “period of FA examination image absence” for which interpolation is possible as identified in step S501 is a time slot TF3302 (from contrast time moment of T1 sec. to contrast time moment of T2 sec.).

Referring back to FIG. 19, the explanation continues.

Upon the end of processing in step S501, the process proceeds to step S502.

Upon proceeding to step S502, the image generation model 2520 identifies the FA examination image that is present immediately before the “period of FA examination image absence” for which interpolation is possible, which has been identified in step S501, and the FA examination image that is present immediately after it. In the example illustrated in FIG. 20, the FA examination image Im3312, which is present immediately before it, and the FA examination image Im3313, which is present immediately after it, are identified in step S502.

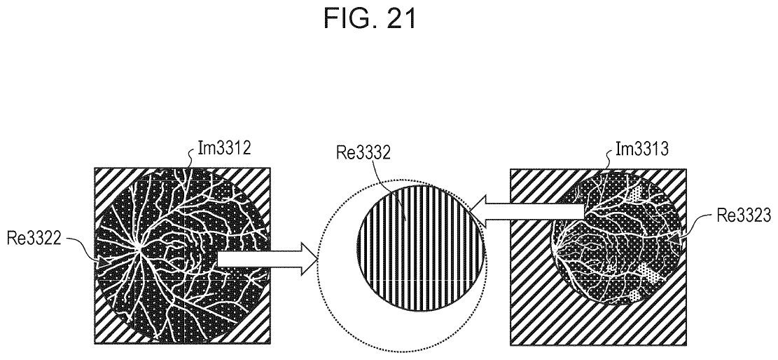

Next, in step S503, the image generation model 2520 finds an effective pixel area that is common to the “immediately-before” FA examination image and the “immediately-after” FA examination image identified in step S502. “Effective pixel area” mentioned here means a pixel area where a contrast effect is depicted. FIG. 21 is a diagram illustrating the third variation example of the third embodiment for explaining the effective pixel area Re3332 that is common to the “immediately-before” FA examination image Im3312 and the “immediately-after” FA examination image Im3313 illustrated in FIG. 20. In FIG. 21, for example, the masked area located around the “immediately-before” FA examination image Im3312 is not an effective pixel area because a contrast effect is not depicted thereat, and the non-masked area located at the center is an effective pixel area Re3322 because a contrast effect is depicted thereat. Similarly, an effective pixel area Re3323 in the “immediately-after” FA examination image Im3313 can be found. Then, in the example illustrated in FIG. 21, the area where the effective pixel area Re3322 of the “immediately-before” FA examination image Im3312 overlaps with the effective pixel area Re3323 of the “immediately-after” FA examination image Im3313 is the common effective pixel area Re3332 found in step S503.

Referring back to FIG. 19, the explanation continues.

Upon the end of processing in step S503, the process proceeds to step S504.

Upon proceeding to step S504, the image generation model 2520 generates an interpolation image. Specifically, the image generation model 2520 generates the interpolation image by using the pixel values of the common effective pixel area Re3332 in the “immediately-before” FA examination image Im3312 and the pixel values of the common effective pixel area Re3332 in the “immediately-after” FA examination image Im3313. In the example illustrated in FIG. 20, the interpolation image is generated by linearly interpolating the FA examination image in the period of FA examination image absence (the time slot TF3302) from the contrast time moment of T1 sec. to the contrast time moment of T2 sec.

Specifically, in step S504 illustrated in FIG. 19, the interpolation image is generated by performing the following processing.

Let Aᵢⱼ be the pixel value of the “immediately-before” FA examination image Im3312 at the pixel coordinates (x,y). Let Bᵢⱼ be the pixel value of the “immediately-after” FA examination image Im3313 at the pixel coordinates (x,y). In this case, the pixel value Lᵢⱼ of the interpolation image at the pixel coordinates (x,y) at the point in time of t sec. can be expressed by the following equation (1):

Iᵢⱼ=(1−α)×Aᵢⱼ+α×Bᵢⱼ (1),

where α=t/(T2−T1) in (1).

Pixel values dealt with as a masked area, at which the pixel values are always zero or so, are applied to areas other than the common effective pixel area Re3332.

Upon the end of processing in step S504, the processing illustrated in the flowchart of FIG. 19 ends. Through the interpolation image generation processing illustrated in the flowchart of FIG. 19, for example, it is possible to generate interpolation images at one-second intervals for the time slot TF3302, which is the period of FA examination image absence in FIG. 20, and add them into the data set.

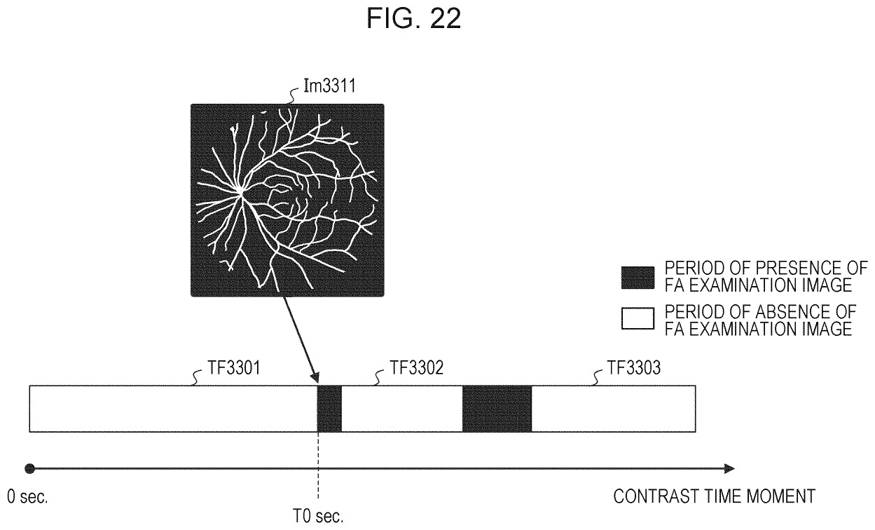

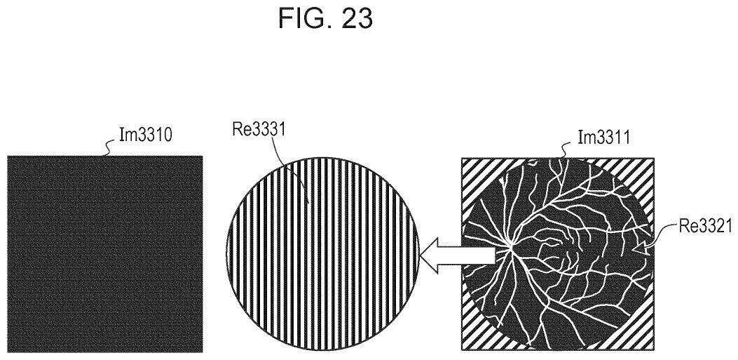

With reference to FIGS. 22 and 23, a further application example will now be described.

FIG. 22 is a diagram illustrating the third variation example of the third embodiment, and illustrating an example of a period of presence of an FA examination image included in teacher data that is used when the image generation model 2520 is trained and a period of absence thereof. FIG. 23 is a diagram illustrating the third variation example of the third embodiment for explaining an effective pixel area Re3331 in a case where the “immediately-after” FA examination image Im3311 illustrated in FIG. 22 is the “shot first” FA examination image in an FA examination. In FIG. 22, let us consider a case where the FA examination image Im3311, which is present immediately after the period of FA examination image absence such as a time slot TF3301, is the “shot first” FA examination image in an FA examination. In this case, as illustrated in FIG. 23, an FA examination image Im3310 which is a pitch-black image (for example, an image blacked out by the same pixel value as that of a masked area) and the entire area of which is an effective pixel area may be set as an FA examination image at the point in time of zero (the contrast time moment of zero) in FIG. 22. Specifically, the FA examination image Im3310 illustrated in FIG. 23 may be set as a virtual FA examination image that is virtually present immediately before the period of FA examination image absence. In the example illustrated in FIG. 23, the effective pixel area Re3331 that is common to the virtual “immediately-before” FA examination image Im3310 and the “immediately-after” FA examination image Im3311 is the same as the effective pixel area Re3321 of the “immediately-after” FA examination image Im3311.

The third variation example of the third embodiment is effective in improving the image generation precision (the likelihood of depicting by the contrast effect image) of the image generation model 2520, which is achieved by augmenting the pieces of teacher data in the data set by performing FA examination image interpolation in the period of FA examination image absence. This makes it possible to desirably acquire an image that depicts a contrast effect corresponding to the contrast time moment at which the operator wants to make an observation, thereby assisting the operator in making a decision in a diagnosis.

### Fourth Embodiment

Next, a fourth embodiment will now be described. In the fourth embodiment described below, description of matters that are the same as those having been described in the first to third embodiments above will be omitted, and matters that are different from those having been described in the first to third embodiments above will be described.

The schematic configuration of an image generation system that includes an image generation apparatus according to the fourth embodiment is the same as the schematic configuration of the image generation system 1 that includes the image generation apparatus 20 according to the first embodiment illustrated in FIG. 1.

The outputting unit 252 according to the fourth embodiment outputs a still-picture contrast effect image group that depicts a contrast effect corresponding to contrast time that includes a predetermined contrast time moment group comprised of plural pieces of contrast time moment on the basis of a medical image that is a still image acquired by the image acquisition unit 251.

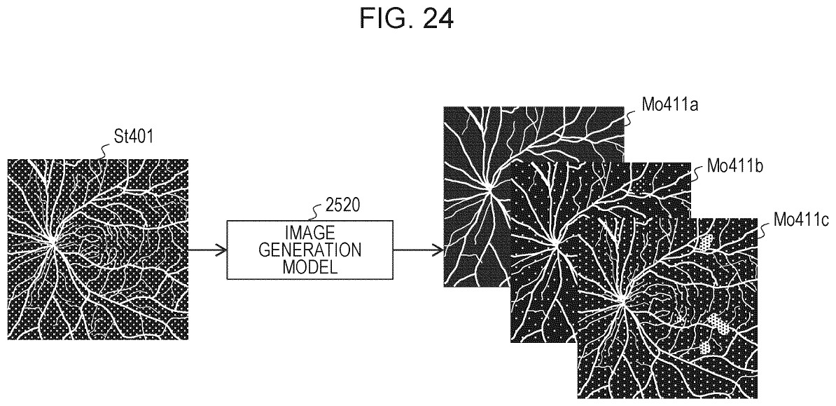

FIG. 24 is a diagram for explaining the concept of the image generation model 2520 of the outputting unit 252 in the image generation apparatus 20 according to the fourth embodiment. In FIG. 24, the same reference signs are assigned to components, etc. that are the same as those illustrated in FIG. 2, and a detailed explanation thereof is omitted.

The outputting unit 252 according to the fourth embodiment includes the image generation model 2520 illustrated in FIG. 24. The image generation model 2520 illustrated in FIG. 24 is an image generation model 2520 that includes an image processing system based on deep learning technology.

The image generation model 2520 illustrated in FIG. 24 receives an input image St401, which is a still-picture medical image. Then, the image generation model 2520 illustrated in FIG. 24 outputs output images Mo411a to Mo411c as a still-picture contrast effect image group that depicts a contrast effect corresponding respectively to a predetermined contrast time moment group comprised of plural pieces of contrast time moment on the basis of the input image St401.

The image generation model 2520 illustrated in FIG. 24 includes the U-Net-based network model 2521 as the image processing system based on deep learning technology, and outputs a contrast effect image group that depicts a contrast effect at a predetermined contrast time moment group comprised of N pieces of contrast time moment. “Predetermined contrast time moment group comprised of N pieces of contrast time moment” is a set of pieces of contrast time moment such as, for example, “30 sec., 60 sec., and 200 sec.” after the reference point in time. The predetermined contrast time moment may preferably be clinically useful contrast time moment. For example, a selection may be made from the following time regarded as important in FA examinations: “before 60 sec. (early contrast phase)” after the reference point in time, “from 60 sec. to 200 sec. (middle contrast phase)” after the reference point in time, “after 200 sec. (late contrast phase)” after the reference point in time, and the like.

The image generation model 2520 illustrated in FIG. 24 inputs the input image St401, which is a still image, after transforming it into a tensor, into the network model (2521). Then, the image generation model 2520 illustrated in FIG. 24 applies still-picture transformation to tensors outputted from the network model (2521) and outputs these still images as the output images Mo411a to Mo411c. In a case where U-Net is adopted as the network model (2521), there is a need to modify the U-Net.

The following is a specific example. Let us consider a case where the shape of the tensor transformed from the input image St401, which is a still image, is “Cᵢₙ× Hᵢₙ×Wᵢₙ” having been explained earlier in the first embodiment. In the network model (2521) with U-Net modification, the number of elements that constitute the input tensor is increased, and shape deformation is performed up to the last layer, thereby outputting a tensor whose shape is “N× Cₒᵤₜ×Hₒᵤₜ× Wₒᵤₜ”. The tensor outputted from the network model (2521) is divided into N tensors each having a shape of “Cₒᵤₜ×Hₒᵤₜ×Wₒᵤₜ”. Then, each of the tensors after the division is transformed into a still image, and the output images Mo411a to Mo411c are outputted from the image generation model 2520 as a contrast effect image group. The tensor shape is not limited to the shape described in the present embodiment. It may be any shape with which the same object can be achieved. Though U-Net is taken as an example in the present embodiment, any other network model with which the same object can be achieved may be adopted.

A data set for training the image generation model 2520 illustrated in FIG. 24, which includes the network model (2521) based on U-Net, will now be described. A data set has a structure of a teacher data group acquired from a plurality of examination targets, wherein an OCTA image that is a still image acquired by taking a shot of the same examination target, and FA examination images (group) captured at one or more pieces of contrast time moment among the predetermined contrast time moment group comprised of N pieces of contrast time moment, are paired to constitute each one piece of teacher data in the group. The examination target is, in the present embodiment, the subject eye.

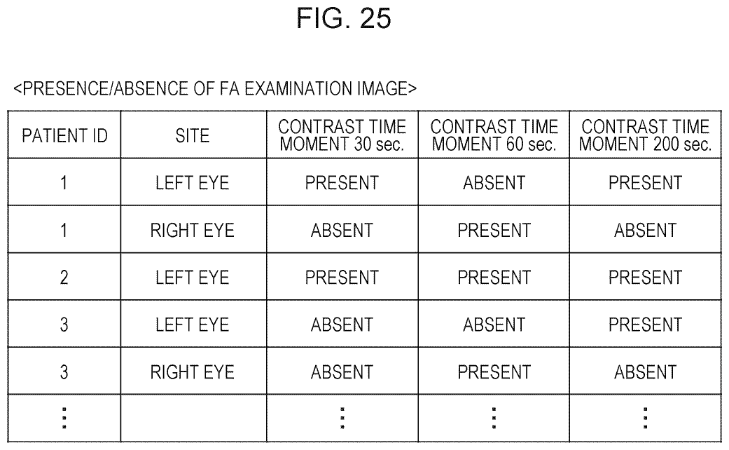

For easier explanation, it is assumed below that the predetermined contrast time moment group comprised of N pieces of contrast time moment is comprised of three pieces of contrast time moment that are “30 sec., 60 sec., and 200 sec.” after the reference point in time. FIG. 25 is a diagram illustrating the fourth embodiment for explaining the presence/absence of FA examination images included in teacher data that is used when the image generation model 2520 is trained. As explained earlier with reference to FIG. 16 in the third embodiment, in an FA examination, time slots during which imaging cannot be performed could exist; therefore, for example, it could happen that gathered FA examination images included in teacher data are such as those illustrated in FIG. 25.

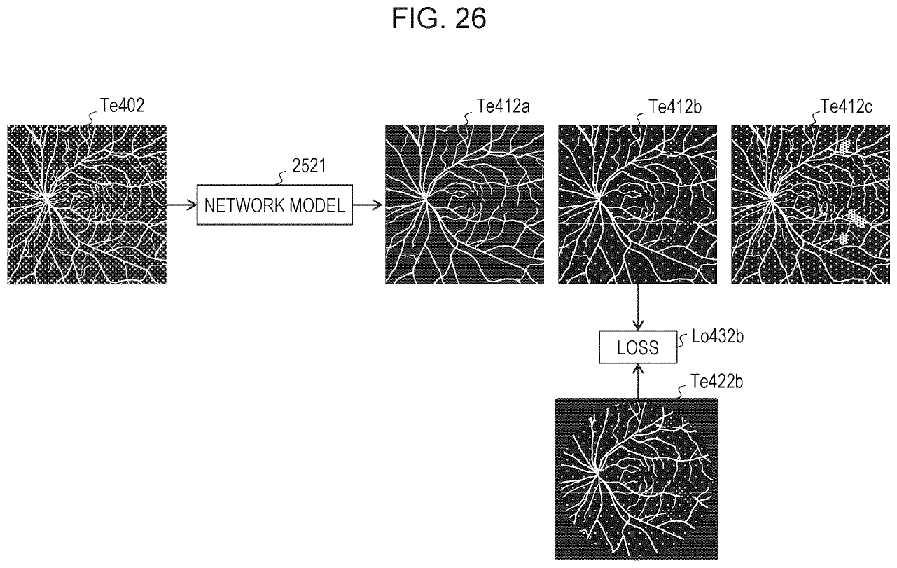

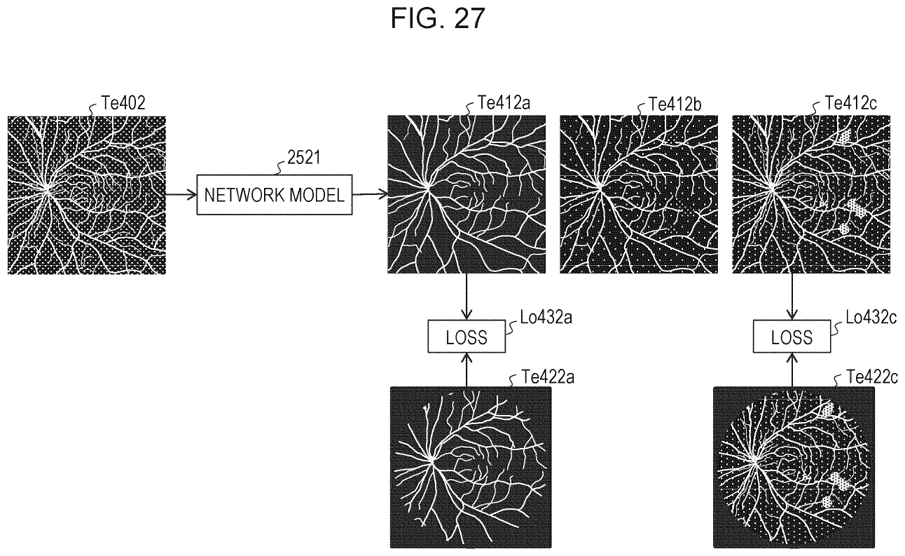

FIGS. 26 and 27 are diagrams for explaining the training of the image generation model 2520 of the outputting unit 252 in the image generation apparatus 20 according to the fourth embodiment. In the present embodiment, the image generation model 2520 of the outputting unit 252 includes the network model 2521 illustrated in FIGS. 26 and 27. With reference to FIGS. 26 and 27, the training of the image generation model 2520 using certain one pair of teacher data, that is, processing for updating the parameters that constitute the network model 2521 included in the image generation model 2520, will now be described.

First, in FIG. 26, an input tensor Te402, which is a tensor transformed from an OCTA image included in teacher data, is inputted into the network model 2521. Upon the input, output tensors Te412a to Te412c, which correspond to three still-picture contrast effect images, are outputted from the network model 2521. Next, the image generation model 2520 calculates a loss group while excluding missing contrast time moment in a still-picture FA examination image group included in the same teacher data, and outputs an average of the loss group as a final loss. For example, if the FA examination image at the contrast time moment of 60 sec. is the sole one included in the teacher data, the processing illustrated in FIG. 26 is performed. That is, in this case, as illustrated in FIG. 26, a loss Lo432b, which is an error between a ground truth tensor Te422b transformed from the FA examination image at the contrast time moment of 60 sec. and an output tensor Te412b corresponding thereto, is calculated and outputted as the final loss. As another pattern, if two FA examination images only, one of which is at the contrast time moment of 30 sec. and the other of which is at the contrast time moment of 200 sec., are included in the teacher data, the processing illustrated in FIG. 27 is performed. That is, in this case, as illustrated in FIG. 27, a loss Lo432a regarding the contrast time moment of 30 sec. and a loss Lo432c regarding the contrast time moment of 200 sec. are calculated similarly, and (Lo432a+Lo432c)/2, which is a value obtained by averaging these two losses, is outputted as the final loss. Finally, the image generation model 2520 updates the parameters that constitute the network model 2521 in such a way as to make the final loss small. This series of update processing is repeated while using a teacher data group assigned for training among the data set until the network model 2521 becomes trained enough.

The image generation model 2520 having been trained through the learning processing described above is capable of outputting a contrast effect image group comprised of a plurality of still-picture contrast effect images depicting a contrast effect having a plausible likelihood based on the teacher data group assigned for training among the data set, upon receiving an input of an OCTA image. Specifically, it is possible to output a contrast effect image group comprised of three still-picture contrast effect images depicting a contrast effect having a plausible likelihood and corresponding to the contrast time moment of 30 sec., 60 sec., and 200 sec. That is, it is possible to output a pseudo contrast image group (contrast effect image group) that resembles FA examination images in a still-picture format depicting a contrast effect corresponding to the contrast time moment of three points in time, like those acquired in FA examinations.

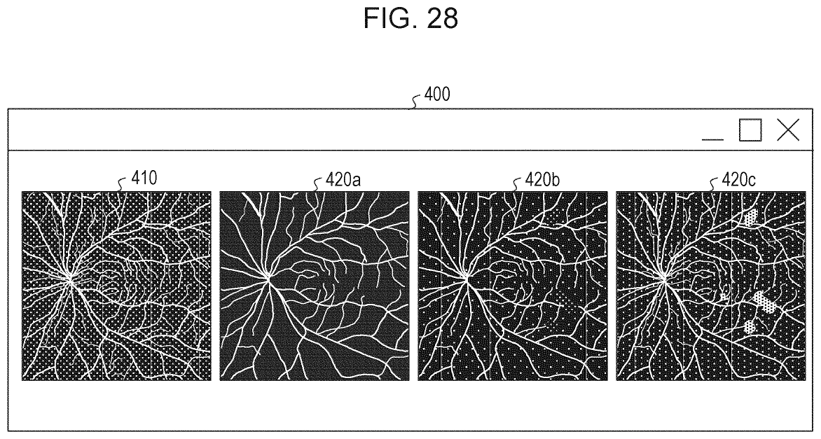

FIG. 28 is a diagram illustrating an example of the GUI screen 400 displayed on the display 230 in the image generation apparatus 20 according to the fourth embodiment.

The display unit 253 performs processing of displaying the GUI screen 400 illustrated in FIG. 28 on the display 230. Specifically, the display unit 253 performs processing of displaying the medical image acquired by the image acquisition unit 251 (in the present embodiment, the OCTA image) in the image display area 410 of the GUI screen 400 illustrated in FIG. 28. In addition, the display unit 253 performs processing of displaying the three contrast effect images outputted from the outputting unit 252 in image display areas 420a to 420c of the GUI screen 400 illustrated in FIG. 28. In the present embodiment, the contrast effect image corresponding to the contrast time moment of 30 sec. is displayed in the image display area 420a, the contrast effect image corresponding to the contrast time moment of 60 sec. is displayed in the image display area 420b, and the contrast effect image corresponding to the contrast time moment of 200 sec. is displayed in the image display area 420c. Therefore, the operator can observe the contrast effect images corresponding to the respective pieces of contrast time moment by viewing the image display areas 420a to 420c of the GUI screen 400.

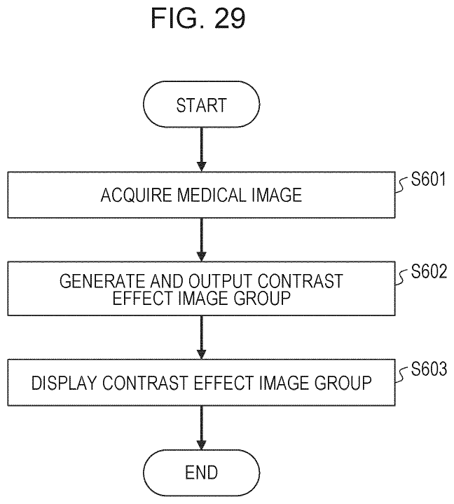

FIG. 29 is a flowchart illustrating an example of processing steps in a method of controlling the image generation apparatus 20 according to the fourth embodiment.

Upon the start of processing illustrated in the flowchart of FIG. 29, first, in step S601, the image acquisition unit 251 acquires an OCTA image, which is a medical image, from the imaging apparatus 10, for example.

Next, in step S602, the outputting unit 252 generates and outputs a contrast effect image group that depicts a contrast effect corresponding to contrast time that includes a predetermined contrast time moment group comprised of plural pieces of contrast time moment on the basis of the OCTA image acquired in step S601. Specifically, in the present embodiment, the outputting unit 252 outputs a group of contrast effect images each of which is a pseudo contrast image that resembles an FA examination image in a still-picture format depicting a contrast effect corresponding to the contrast time moment in the predetermined contrast time moment group.

Next, in step S603, the display unit 253 displays the OCTA image acquired in step S601 in the image display area 410 of the GUI screen 400 illustrated in FIG. 28 and displays the contrast effect image group outputted in step S602 in the image display areas 420a to 420c thereof. That is, as shown in the GUI screen 400 illustrated in FIG. 28, the OCTA image acquired in step S601 and the contrast effect image group outputted in step S602 are displayed in a line.

Upon the end of processing in step S603, the processing illustrated in the flowchart of FIG. 29 ends.

As explained above, in the image generation apparatus 20 according to the fourth embodiment, the image acquisition unit 251 acquires an OCTA image, which is a medical image, from the imaging apparatus 10, for example. Then, the outputting unit 252 outputs a contrast effect image group that depicts a contrast effect corresponding to plural pieces of contrast time moment (a pseudo contrast image group that resembles FA examination images in a still-picture format) on the basis of the OCTA image acquired by the image acquisition unit 251.

With this configuration, it is possible to desirably acquire an image group that depicts a contrast effect corresponding to contrast time that includes contrast time moment of a certain plurality of points in time. This makes it possible to desirably acquire an FA-examination-image-like image group that depicts a contrast effect corresponding to the contrast time moment group at each in which the operator wants to make an observation, thereby assisting the operator in making a decision in a diagnosis. Moreover, as compared with a case where a contrast effect image in a moving-picture format is outputted, the image generation apparatus 20 according to the fourth embodiment makes it possible to observe, at a time, the contrast effect image group for the contrast time moment group that is useful for making a diagnosis, and thus offers higher time efficiency. Furthermore, the image generation apparatus 20 according to the fourth embodiment makes the burden of creating a data set lighter because it suffices to gather, as teacher data, only images related to the contrast time moment group at each in which the operator wants to make an observation. Variation Example of Fourth Embodiment

Next, a variation example of the fourth embodiment described above will now be described.

The outputting unit 252 according to the fourth embodiment may include an image generation model group comprised of a plurality of image generation models, and the image generation model 2520, meaning each of them, may output a pseudo contrast effect image that resembles an FA examination image in a still-picture format depicting a contrast effect corresponding to one piece of contrast time moment among the pieces of contrast time moment. That is, in the variation example of the fourth embodiment, the image generation model 2520 that is each of the plurality of image generation models in the group is configured to receive an input of a single OCTA image and output a contrast effect image for the corresponding one piece of contrast time moment.

### Fifth Embodiment

Next, a fifth embodiment will now be described. In the fifth embodiment described below, description of matters that are the same as those having been described in the first to fourth embodiments above will be omitted, and matters that are different from those having been described in the first to fourth embodiments above will be described.

The schematic configuration of an image generation system that includes an image generation apparatus according to the fifth embodiment is the same as the schematic configuration of the image generation system 1 that includes the image generation apparatus 20 according to the second embodiment illustrated in FIG. 11.

In the fifth embodiment, the imaging conditions acquired by the imaging condition acquisition unit 254 include other conditions in addition to contrast time that includes contrast time moment, and it is possible to influence a contrast effect image that the outputting unit 252 outputs, correspondingly to said other conditions included in the imaging conditions. Said other conditions included in the imaging conditions include information related to an FA examination that is one or more of the following: yes/no (with/without) of individual image processing (optional image-quality enhancement processing, etc.) of an FA examination image, imaging angle of field of an FA examination image, subject information (gender, age, imaging site, yes/no (with/without) of medical treatment, etc.), model of an FA examination apparatus, etc.

The imaging condition acquisition unit 254 according to the fifth embodiment acquires imaging conditions that include, in addition to contrast time that includes contrast time moment of at least one point in time, other conditions including one or more of the above-described information related to an FA examination. That is, the imaging condition acquisition unit 254 according to the fifth embodiment acquires the above-described imaging conditions that include contrast time and further includes different information other than the contrast time. The outputting unit 252 according to the fifth embodiment outputs, on the basis of a medical image that is a still image acquired by the image acquisition unit 251 and on the basis of the imaging conditions acquired by the imaging condition acquisition unit 254, a contrast effect image that is a still image that depicts a contrast effect. For this processing, the medical image, and, as the imaging conditions, the contrast time and the information other than the contrast time, are inputted into the image generation model 2520 of the outputting unit 252.

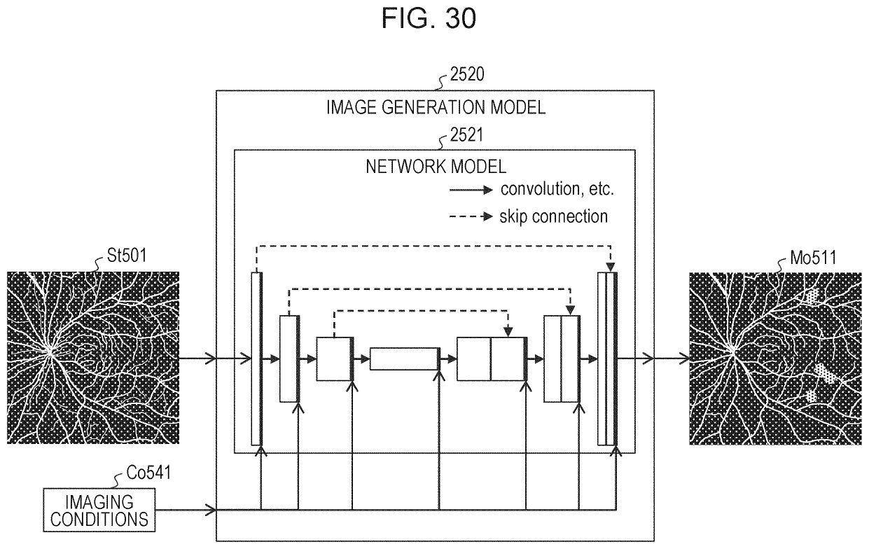

FIG. 30 is a diagram for explaining the concept of the image generation model 2520 of the outputting unit 252 in the image generation apparatus 20 according to the fifth embodiment. In FIG. 30, the same reference signs are assigned to components, etc. that are the same as those illustrated in FIGS. 2, 14, and 15, and a detailed explanation thereof is omitted.

The outputting unit 252 according to the fifth embodiment includes the image generation model 2520 illustrated in FIG. 30. The image generation model 2520 illustrated in FIG. 30 includes the U-Net-based network model 2521 as the image processing system based on deep learning technology. Though U-Net is taken as an example in the present embodiment, any other network model with which the same object can be achieved may be adopted.

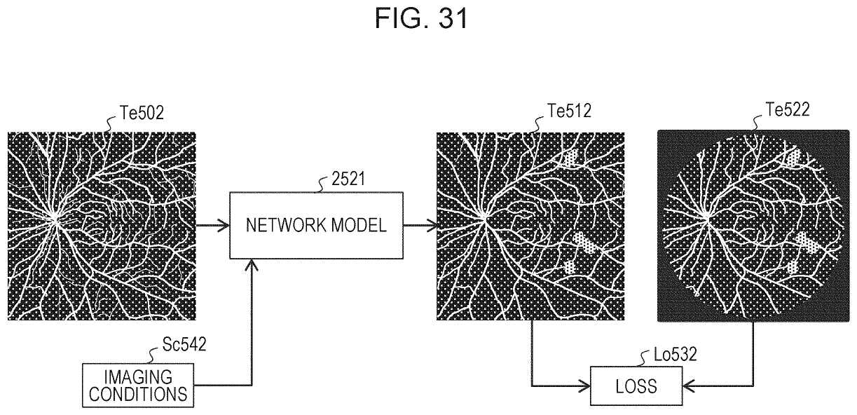

FIG. 31 is a diagram for explaining the training of the image generation model 2520 of the outputting unit 252 in the image generation apparatus 20 according to the fifth embodiment. In FIG. 31, the same reference signs are assigned to components, etc. that are the same as those illustrated in FIG. 30, and a detailed explanation thereof is omitted.

The image generation model 2520 illustrated in FIG. 30 receives an input image St501, which is a still-picture medical image, and imaging conditions Co541 as inputs, and generates a still-picture contrast effect image that depicts a contrast effect on the basis of the input image St501. Specifically, the image generation model 2520 illustrated in FIG. 30 inputs, into the network model 2521, an input tensor Te502 illustrated in FIG. 31, which is transformed from the input image St501 illustrated in FIG. 30, and a tensor transformed from the imaging conditions Co541 illustrated in FIG. 30 (Sc542). Then, the image generation model 2520 illustrated in FIG. 30 applies still-picture transformation to a tensor outputted from the network model 2521 and outputs an output image Mo511 as a contrast effect image.

In a case where U-Net is adopted as the network model 2521, there is a need to modify the U-Net. Though the method of this modification is roughly the same as that of the third embodiment, there is a difference. The difference from the third embodiment is as follows.

Specifically, a scalar value group Sc542 that represents the imaging conditions Co541 is given to at least one tensor space axis among the number of channels, height, and width of at least one of tensors generated in the intermediate layer of the network model 2521. The scalar value group Sc542 is a set of scalar values determined on the basis of pieces of information related to an FA examination included in the imaging conditions Co541. For example, for information that can be expressed by means of a continuous value, such as, contrast time moment, age, etc., a scalar value is set through division by a constant, similarly to the third embodiment. For information that can be expressed by means of a Boolean value, such as the yes/no of individual image processing, the yes/no of medical treatment, etc., for example, a scalar value of 0 for False and 1 for True is set. For information that can be expressed as category, such as gender, imaging site, imaging angle of field (30°, 55°, etc.), model of an FA examination apparatus, etc., for example, a scalar value is set through division of the corresponding category value by a constant. The following is a specific example. Suppose that the category value of gender information for male is 0 and for those unknown and others is 2; in this case, the division may be performed using 2, which is the maximum of the category values, as the constant to obtain a scalar value of 0, 0.5, and 1 for each. The object here is to input an information group related to an FA examination into the network model 2521 and, therefore, using the method described above for conversion into scalar values is not necessarily needed. For example, although age has been treated as continuous values in the above-described example of conversion into scalar values while assuming that age information is included as the information related to an FA examination, categorization as discrete values may be performed instead. Alternatively, age may be treated as age groups, and conversion into scalar values may be performed on the basis of category values such as “20s”, “30s”, “40s”, and the like. As a specific method of the giving, for example, let us consider a case where the original shape of a tensor before the scalar value group Sc542 is given thereto is “B×C×H×W”, and the number of pieces of information included in the imaging conditions Co541 (that is, the number of values in the scalar value group Sc542) is M. In this case, the number of channels is extended into a shape of “B× (C+M)× H×W”. Then, processing of filling each one channel region of the extended tensor region with each scalar value included in the scalar value group Sc542 is added, and, in addition, the structure of the network model 2521 is altered so as to make it possible to process the extended tensor. Alternatively, if the number of channels is M+1 or more, the values of an arbitrary tensor region corresponding to M channels may be filled with the respective scalar values included in the scalar value group Sc542, instead of the tensor extension. The object here is to input an information group related to an FA examination into the network model 2521 and, therefore, using the method described above for inputting the scalar value group Sc542 into the network model 2521 is not necessarily needed. For example, the respective scalar values included in the scalar value group Sc542 may be given to different tensors of the tensor group generated in the intermediate layer of the network model 2521.

A data set for training the image generation model 2520, which includes the above-described U-Net-based network model 2521, will now be described. A data set has a structure of a teacher data group acquired from a plurality of examination targets, wherein an OCTA image that is a still image acquired by taking a shot of the same examination target, an FA examination image, and an imaging condition that at least includes the contrast time moment of the FA examination image are “paired” to constitute each one piece of teacher data in the group. The examination target is, in the present embodiment, the subject eye. With reference to FIG. 31, the training of the image generation model 2520 using certain one pair of teacher data, that is, processing for updating the parameters that constitute the network model 2521 included in the image generation model 2520, will now be described.

First, in FIG. 31, the input tensor Te502, which is a tensor transformed from the OCTA image included in the teacher data, and the scalar value group Sc542, which represents the imaging conditions Co541 included in the same teacher data, are inputted into the network model 2521. Upon the input, an output tensor Te512, which corresponds to the contrast effect image that is a still image, is outputted from the network model 2521. Next, the image generation model 2520 calculates a loss Lo532, which is an error of the output tensor Te512 compared with a ground truth tensor Te522, which is a tensor transformed from the FA examination image that is a still image included in the same teacher data. Finally, the image generation model 2520 updates the parameters that constitute the network model 2521 in such a way as to make the loss Lo532 small. This series of update processing is repeated while using a teacher data group assigned for training among the data set until the network model 2521 becomes trained enough.

The image generation model 2520 having been trained through the learning processing described above is capable of outputting a still-picture contrast effect image that depicts a contrast effect having a plausible likelihood based on the teacher data group assigned for training among the data set, upon receiving an input of an OCTA image. That is, it is possible to output a pseudo contrast image (contrast effect image) that resembles an FA examination image in a still-picture format depicting a contrast effect corresponding to the designated contrast time moment, like those acquired in FA examinations.

Processing steps in a method of controlling the image generation apparatus 20 according to the fifth embodiment are the same as the processing steps illustrated in the flowchart of FIG. 13, which relates to the method of controlling the image generation apparatus 20 according to the second embodiment. With reference to the flowchart of FIG. 13, the processing steps in the method of controlling the image generation apparatus 20 according to the fifth embodiment will now be described.

In the fifth embodiment, upon the start of processing illustrated in the flowchart of FIG. 13, first, in step S301, the image acquisition unit 251 acquires an OCTA image, which is a medical image, from the imaging apparatus 10, for example.

Next, in step S302, the imaging condition acquisition unit 254 acquires imaging conditions that include contrast time that includes contrast time moment of at least one point in time and information other than the contrast time.

Next, in step S303, the outputting unit 252 generates and outputs a contrast effect image that depicts a contrast effect on the basis of the OCTA image acquired in step S301 and on the basis of the imaging conditions acquired in step S302. Specifically, in the present embodiment, the outputting unit 252 outputs a contrast effect image that is a pseudo contrast image that resembles an FA examination image in a still-picture format depicting a contrast effect.

Next, in step S304, the display unit 253 displays the OCTA image acquired in step S301 in the image display area 410 of the GUI screen 400 illustrated in FIG. 12 and displays the contrast effect image outputted in step S303 in the image display area 420 thereof.

Upon the end of processing in step S304, the processing illustrated in the flowchart of FIG. 13 ends.

As explained above, in the image generation apparatus 20 according to the fifth embodiment, the image acquisition unit 251 acquires an OCTA image, which is a medical image, from the imaging apparatus 10, for example. The imaging condition acquisition unit 254 acquires imaging conditions that include contrast time that includes contrast time moment of at least one point in time and information other than the contrast time. Then, the outputting unit 252 outputs a contrast effect image that depicts a contrast effect (a pseudo image that resembles an FA examination image in a still-picture format) on the basis of the OCTA image acquired by the image acquisition unit 251 and on the basis of the imaging conditions acquired by the imaging condition acquisition unit 254.

With this configuration, it is possible to desirably acquire an FA-examination-image-like image that depicts a contrast effect corresponding to the contrast time moment at which the operator wants to make an observation, thereby assisting the operator in making a decision in a diagnosis. Furthermore, the image generation apparatus 20 according to the fifth embodiment is capable of influencing a contrast effect image, correspondingly to other conditions included in the imaging conditions, namely, the information other than the contrast time, in comparison with the third embodiment, for example.

### Variation Example of Fifth Embodiment

Next, a variation example of the fifth embodiment described above will now be described.

The imaging condition acquisition unit 254 according to the fifth embodiment described above may include, as the imaging conditions, in addition to information related to an FA examination, information related to an OCTA examination, and the information related to an OCTA examination may be included in the teacher data, too. The information related to an OCTA examination includes the model of an OCTA examination apparatus, the yes/no of individual image processing of an OCTA image, a depth range for OCTA image generation (a superficial layer, a deep layer, an outer layer, a choroidal vascular network, etc.), and the imaging angle of field of an OCTA image. The information related to an OCTA examination further includes the resolution of an OCTA image and the scan mode (Cross, Radial) of an OCTA image.

With the variation example of the fifth embodiment, the information related to an OCTA examination can also be reflected in the image generation processing performed by the image generation apparatus 20, and it is thus possible to acquire a contrast effect image that depicts a contrast effect on the basis of more detailed features of the inputted OCTA image. This makes it possible to desirably acquire a contrast effect image that depicts a contrast effect corresponding to the contrast time that includes the contrast time moment at which the operator wants to make an observation, thereby assisting the operator in making a decision in a diagnosis.

### Sixth Embodiment

Next, a sixth embodiment will now be described. In the sixth embodiment described below, description of matters that are the same as those having been described in the first to fifth embodiments above will be omitted, and matters that are different from those having been described in the first to fifth embodiments above will be described.

The schematic configuration of an image generation system that includes an image generation apparatus according to the sixth embodiment is the same as the schematic configuration of the image generation system 1 that includes the image generation apparatus 20 according to the second embodiment illustrated in FIG. 11.

The imaging condition acquisition unit 254 according to the sixth embodiment acquires imaging conditions that include, in addition to contrast time that includes contrast time moment of at least one point in time, information other than the contrast time. In the present embodiment, the information other than the contrast time included in the imaging conditions includes one or more pieces of information related to an OCTA examination or an FA examination and interpretable as category.

The outputting unit 252 according to the sixth embodiment includes an image generation model group comprised of a plurality of image generation models 2520. The image generation models in the image generation model group are constructed to correspond to the types of the category-interpretable information included in the imaging conditions acquired by the imaging condition acquisition unit 254, with differences in quality about contrast effect depiction. For example, in a case where “depth range information (a superficial layer, a deep layer, an outer layer, a choroidal vascular network, etc.)” for OCTA image generation is included as the information related to an OCTA examination in the imaging conditions, the outputting unit 252 includes a plurality of image generation models categorized on a depth-range-by-depth-range basis. Specifically, for example, the image generation model group includes “an image generation model for a superficial layer”, “an image generation model for a deep layer”, “an image generation model for an outer layer”, “an image generation model for a choroidal vascular network”, etc.

The outputting unit 252 according to the sixth embodiment selects an appropriate image generation model from among the plurality of image generation models 2520 on the basis of the information other than the contrast time included in the imaging conditions. Then, by using the selected image generation model, the outputting unit 252 according to the sixth embodiment outputs a contrast effect image on the basis of the medical image acquired by the image acquisition unit 251 and on the basis of the imaging conditions acquired by the imaging condition acquisition unit 254. Specifically, the outputting unit 252 according to the sixth embodiment selects an appropriate image generation model on the basis of the above-described depth range information included in the imaging conditions, and performs processing for generating a contrast effect image.

As another example, in a case where “the yes/no (with/without) of individual image processing (optional image-quality enhancement processing, etc.)” is included as the information related to an FA examination in the imaging conditions, the outputting unit 252 includes two image generation models 2520 respectively for the yes of individual image processing and the no of individual image processing. Specifically, these two image generation models are: “the image generation model 2520 with individual image processing” and “the image generation model 2520 without individual image processing”. In this case, the outputting unit 252 selects an appropriate image generation model 2520 in accordance with the yes/no of individual image processing included in the imaging conditions, and performs processing for generating a contrast effect image. In some cases, a continuous value included in the imaging conditions can be interpreted as category. For example, the category may be determined depending on the value of contrast time moment, such as “before 100 sec.”, “after 100 sec. inclusive, but before 200 sec.”, “after 200 sec. inclusive”. In a case where new category information can be generated from contrast time moment as in this example, the contrast time moment only suffices as the information included in the imaging conditions.

Each of the plurality of image generation models 2520 in the image generation model group includes the network model 2521 having been trained using a data set suited for the imaging conditions in which it is used. Specifically, the structure of a data set used for training the network model 2521 in a case where the “depth range information” for generating an OCTA image is “a superficial layer” is as follows: a teacher data group acquired from a plurality of examination targets, wherein an OCTA image that is a still image acquired by taking a shot of the same examination target for the depth range “superficial layer”, an FA examination image, and an imaging condition that at least includes the contrast time moment of the FA examination image are “paired” to constitute each one piece of teacher data in the group. The examination target is, in the present embodiment, the subject eye.

There is no need to input an imaging condition that is a factor resulting in selecting the image generation model 2520 (hereinafter will be referred to as “imaging condition for image generation model selection”) into the selected image generation model 2520. For this reason, imaging conditions that exclude the imaging condition for image generation model selection are inputted into the image generation model 2520. This means that the imaging conditions include contrast time that includes contrast time moment of at least one point in time and other imaging conditions required by the selected image generation model 2520. For example, there is no need to input “depth range information” into the “image generation model for a superficial layer” described above, which is used in a case where the depth range information is “a superficial layer”. Therefore, the imaging conditions inputted into the “image generation model for a superficial layer” do not include the “depth range information” and do include the contrast time that includes contrast time moment of at least one point in time.

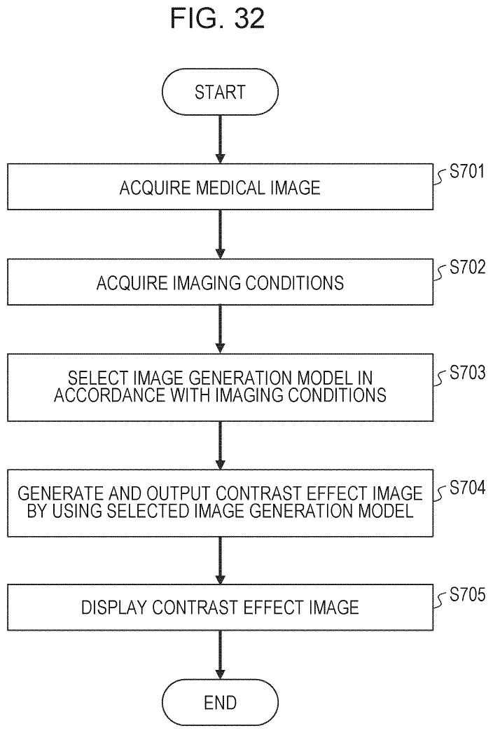

FIG. 32 is a flowchart illustrating an example of processing steps in a method of controlling the image generation apparatus 20 according to the sixth embodiment.

Upon the start of processing illustrated in the flowchart of FIG. 32, first, in step S701, the image acquisition unit 251 acquires an OCTA image, which is a medical image, from the imaging apparatus 10, for example.

Next, in step S702, the imaging condition acquisition unit 254 acquires imaging conditions that include, in addition to contrast time that includes contrast time moment of at least one point in time, information other than the contrast time. In the present embodiment, the information other than the contrast time included in the imaging conditions includes one or more pieces of information related to an OCTA examination or an FA examination and interpretable as category.

Next, in step S703, the outputting unit 252 selects an appropriate image generation model from among the plurality of image generation models 2520 on the basis of the information other than the contrast time included in the imaging conditions (information that is interpretable as category).

Next, in step S704, by using the image generation model 2520 selected in step S703, the outputting unit 252 generates and outputs a contrast effect image that depicts a contrast effect on the basis of the OCTA image acquired in step S701. Specifically, in the present embodiment, the outputting unit 252 outputs a contrast effect image that is a pseudo contrast image that resembles an FA examination image in a still-picture format.

Next, in step S705, the display unit 253 displays the OCTA image acquired in step S701 in the image display area 410 of the GUI screen 400 illustrated in FIG. 12 and displays the contrast effect image outputted in step S704 in the image display area 420 thereof.

Upon the end of processing in step S705, the processing illustrated in the flowchart of FIG. 32 ends.

As explained above, in the image generation apparatus 20 according to the sixth embodiment, the image acquisition unit 251 acquires an OCTA image, which is a medical image, from the imaging apparatus 10, for example. The imaging condition acquisition unit 254 acquires imaging conditions that include, in addition to contrast time that includes contrast time moment of at least one point in time, information other than the contrast time. Then, the outputting unit 252 selects an appropriate image generation model from among the plurality of image generation models 2520 on the basis of the information other than the contrast time included in the imaging conditions (information that is interpretable as category). Then, by using the selected image generation model 2520, the outputting unit 252 outputs a contrast effect image that depicts a contrast effect on the basis of the OCTA image acquired by the image acquisition unit 251.

With this configuration, it is possible to desirably acquire an FA-examination-image-like image that depicts a contrast effect corresponding to the contrast time moment at which the operator wants to make an observation, thereby assisting the operator in making a decision in a diagnosis. Furthermore, since the image generation apparatus 20 according to the sixth embodiment is capable of performing switching among the image generation models 2520 according to the imaging conditions, consequently, it is possible to increase the possibility of acquiring a contrast effect image that depicts a contrast effect that more closely resembles a real contrast image.

### First Variation Example of Sixth Embodiment

Next, as a variation example of the sixth embodiment described above, a first variation example of the sixth embodiment will now be described.

Though the outputting unit 252 according to the sixth embodiment described above includes an image generation model group comprised of a plurality of image generation models 2520, the following variation example can be applied thereto. Specifically, instead of selecting the image generation model 2520 on the basis of the information that is interpretable as category included in the imaging conditions, all of the image generation models in the group may output contrast effect images respectively. In the first variation example of the sixth embodiment, since the selection of the image generation model 2520 is not performed, it is unnecessary that the imaging conditions described above should include the “information that is interpretable as category”.

The plurality of contrast effect images outputted by the plurality of image generation models can be displayed on the GUI screen 400 or be stored in the storage circuit 240 for use in other processing. Furthermore, the plurality of contrast effect images outputted by the plurality of image generation models can be transferred for use to any other non-illustrated apparatus via the NW interface 210 and the network 30.

### Second Variation Example of Sixth Embodiment

Next, as another variation example of the sixth embodiment described above, a second variation example of the sixth embodiment will now be described.

Though the outputting unit 252 according to the sixth embodiment described above includes an image generation model group comprised of a plurality of image generation models 2520, the following variation example can be applied thereto. Specifically, instead of including the image generation model group, the outputting unit 252 may include a single image generation model 2520 capable of outputting a contrast effect image group corresponding to all of the category values defined in the “information that is interpretable as category” included in the imaging conditions.

For example, the following case will now be described: a case where “a superficial layer”, “a deep layer”, “an outer layer”, and “a choroidal vascular network” are defined as the category values corresponding to the “depth range information” having been described in the sixth embodiment. In this case, in the second variation example of the sixth embodiment, the image generation model 2520 of the outputting unit 252 is capable of outputting contrast effect images respectively for the depth ranges of “a superficial layer”, “a deep layer”, “an outer layer”, and “a choroidal vascular network”. In the image generation processing performed by the outputting unit 252, a contrast effect image group corresponding to “a superficial layer”, “a deep layer”, “an outer layer”, and “a choroidal vascular network” respectively is outputted in accordance with at least the contrast time moment included in the imaging conditions. In this example, since the selection of the image generation model 2520 is not performed, it is unnecessary that the imaging conditions should include the “depth range information”, which is the “information that is interpretable as category”. Alternatively, the imaging conditions may include the “depth range information”, and the image generation model 2520 described above may perform processing to output only the contrast effect image that corresponds to the “depth range information”.

### Seventh Embodiment

Next, a seventh embodiment will now be described. In the seventh embodiment described below, description of matters that are the same as those having been described in the first to sixth embodiments above will be omitted, and matters that are different from those having been described in the first to sixth embodiments above will be described.

The schematic configuration of an image generation system that includes an image generation apparatus according to the seventh embodiment is the same as the schematic configuration of the image generation system 1 that includes the image generation apparatus 20 according to the second embodiment illustrated in FIG. 11.

To put it briefly, the outputting unit 252 according to the seventh embodiment receives an input of a radiological image that is a three-dimensional image as a medical image. Then, the outputting unit 252 according to the seventh embodiment outputs a contrast effect image that is a pseudo contrast image that resembles a contrast 4DCT image in a moving image format depicting a contrast effect on the basis of the radiological image.

The image acquisition unit 251 according to the seventh embodiment acquires a radiological image that is a three-dimensional image as a medical image that is a still image acquired by taking a shot of the target of examination by the imaging apparatus 10. The medical image according to the present embodiment, though a three-dimensional CT image is specifically assumed, may be any other kind of a radiological image acquired by the imaging apparatus 10. In the present embodiment, it is sufficient as long as a radiological image can be acquired at the imaging apparatus 10. Therefore, for example, the imaging apparatus 10 may be replaced with an image management system that stores and manages radiological images.

The outputting unit 252 according to the seventh embodiment includes one or more image generation models 2520. The image generation models 2520 may be constructed to correspond to the types of the category-interpretable information included in the imaging conditions acquired by the imaging condition acquisition unit 254, with differences in quality about contrast effect depiction. For example, in a case where “imaging site information (head, chest, abdomen, etc.)” is included as information related to a CT examination in the imaging conditions, the outputting unit 252 includes the plurality of image generation models 2520 in the image generation model group categorized on an imaging-site-by-imaging-site basis. Specifically, for example, the image generation model group here includes “an image generation model for the head”, “an image generation model for the chest”, “an image generation model for the abdomen”, etc.

The outputting unit 252 according to the seventh embodiment selects the image generation model 2520 in accordance with the imaging site information included in the imaging conditions, and performs image generation processing to output a contrast effect image that is a still image. Moreover, in a case where an imaging condition group comprised of a plurality of imaging conditions is designated, the outputting unit 252 according to the seventh embodiment outputs a contrast effect image group that is a plurality of still images correspondingly to the respective imaging conditions. Furthermore, the outputting unit 252 according to the seventh embodiment outputs a contrast effect image that is a moving image using the contrast effect image group as moving-picture frame images. The moving-picture contrast effect image generated here is a three-dimensional moving image, and is a pseudo contrast image that resembles a contrast 4DCT image. As an example of interpreting the value included in the imaging condition as a category value, for example, the category may be determined according to the value of the age of the subject, such as “teens and younger”, “20s to 30s”, “40s and older”, etc.

Each of the plurality of image generation models 2520 in the image generation model group includes the network model 2521 having been trained using a data set suited for the imaging conditions in which it is used. Specifically, the structure of a data set used for training the network model 2521 in a case where the “imaging site information” is “head” is as follows: a teacher data group acquired from a plurality of subjects, wherein a CT image acquired by imaging the “head” of the same examination target, a contrast CT image, and an imaging condition that at least includes the contrast time moment of the contrast CT image are “paired” to constitute each one piece of teacher data in the group.

The imaging condition acquisition unit 254 according to the seventh embodiment acquires an imaging condition group while changing contrast time moment in such a way as to correspond to a predetermined contrast-time-moment-based period (contrast time). For example, suppose that the operator wants to observe a contrast effect at one-second intervals with the predetermined contrast-time-moment-based period designated as “from 0 sec. to 1000 sec.”; in this case, a group comprised of one thousand one imaging conditions (contrast time moment) that are generated while changing the contrast time moment to 1, 2, . . . , 1000 sec. is acquired. The imaging conditions may include information that is interpretable as category such as “imaging site information”.

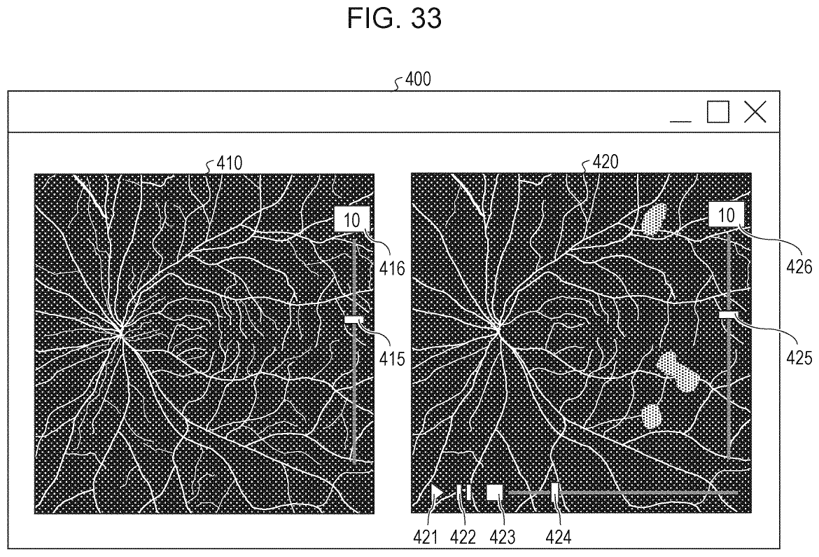

The display unit 253 according to the seventh embodiment displays, in the form of a GUI screen, the contrast effect image outputted from the outputting unit 252 in such a manner that the operator can observe it easily. FIG. 33 is a diagram illustrating an example of the GUI screen 400 displayed on the display 230 in the image generation apparatus 20 according to the seventh embodiment. In FIG. 33, the same reference signs are assigned to components, etc. that are the same as those illustrated in FIG. 5, and a detailed explanation thereof is omitted. The display unit 253 performs processing of displaying the medical image acquired by the image acquisition unit 251 (in the present embodiment, the radiological image) in the image display area 410 of the GUI screen 400 illustrated in FIG. 33. In addition, the display unit 253 performs processing of displaying the contrast effect image outputted from the outputting unit 252 in the image display area 420 of the GUI screen 400 illustrated in FIG. 33. In particular, in a case where a pseudo contrast effect image that resembles a contrast 4DCT image is outputted from the outputting unit 252, the display unit 253 may perform the following display. In this case, as illustrated in FIG. 33, the display unit 253 can display a tomographic position operation slider 425 for operating the three-dimensional image, and a text box 426 thereof, too, in addition to GUI screen components (421 to 424) that enable a play operation and a seek operation of the moving image. In addition, similarly, a slider 415 and a text box 416 thereof can be displayed in the image display area 410 of the GUI screen 400 illustrated in FIG. 33.

Processing steps in a method of controlling the image generation apparatus 20 according to the seventh embodiment are the same as the processing steps illustrated in the flowchart of FIG. 18, which relates to the method of controlling the image generation apparatus 20 according to the first variation example of the third embodiment. With reference to the flowchart of FIG. 18, the processing steps in the method of controlling the image generation apparatus 20 according to the seventh embodiment will now be described.

In the seventh embodiment, upon the start of processing illustrated in the flowchart of FIG. 18, first, in step S401, the image acquisition unit 251 acquires a medical image from the imaging apparatus 10, for example. In the seventh embodiment, a three-dimensional CT image is acquired as a medical image.

Next, in step S402, the imaging condition acquisition unit 254 acquires an imaging condition group (contrast time moment group) while changing contrast time moment in such a way as to correspond to a predetermined contrast-time-moment-based period (contrast time).

Next, in step S403, the outputting unit 252 outputs a contrast effect image group corresponding respectively to the imaging condition group acquired in step S402, on the basis of the three-dimensional CT image acquired in step S401. Specifically, in step S403, the outputting unit 252 outputs a contrast effect image group, each being a pseudo contrast image that resembles a contrast CT image in a still-picture format depicting a contrast effect corresponding to the imaging condition group (contrast time moment group) acquired in step S402.

Next, in step S404, the outputting unit 252 outputs a contrast effect image that is a moving image using the contrast effect image group outputted in step S403 as moving-picture frame images.

Next, in step S405, the display unit 253 displays the CT image acquired in step S401 in the image display area 410 of the GUI screen 400 illustrated in FIG. 33 and displays the contrast effect image that is the moving image outputted in step S404 in the image display area 420 thereof.

Upon the end of processing in step S405, the processing illustrated in the flowchart of FIG. 18 ends.

The seventh embodiment makes it possible to, based on a CT image, acquire a contrast CT image in a moving-picture format that makes it possible to observe time-lapse changes in contrast effect, that is, a pseudo image (contrast effect image) that resembles a contrast 4DCT image. This makes it possible to desirably acquire a contrast-4DCT-like image corresponding to the contrast time moment at which the operator wants to make an observation, thereby assisting the operator in making a decision in a diagnosis.

### Eighth Embodiment

Next, an eighth embodiment will now be described. In the eighth embodiment described below, description of matters that are the same as those having been described in the first to seventh embodiments above will be omitted, and matters that are different from those having been described in the first to seventh embodiments above will be described.

In the first to seventh embodiments, a configuration in which the image generation apparatus 20 is provided as a generator has been described. In the eighth embodiment, a configuration in which an image generation model generator is provided will be described.

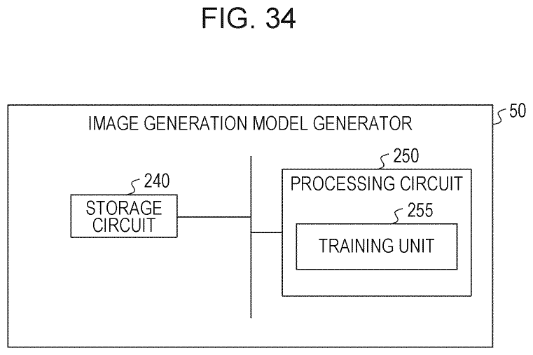

FIG. 34 is a diagram illustrating an example of a schematic configuration of an image generation model generator 50 according to the eighth embodiment. In FIG. 34, the same reference signs are assigned to components, etc. that are the same as those illustrated in FIGS. 1 and 11, and a detailed explanation thereof is omitted.

As illustrated in FIG. 34, the image generation model generator 50 includes the storage circuit 240 and the processing circuit 250. Though an apparatus that includes the storage circuit 240 and the processing circuit 250 is configured as the image generation model generator 50 in FIG. 34, it may be configured as an image generation apparatus 50 similarly to the first to seventh embodiments described above.

The processing circuit 250 illustrated in FIG. 34 controls the operation of the image generation model generator 50 in a central manner, and perform various kinds of processing. As illustrated in FIG. 34, the processing circuit 250 includes a training unit 255. In the present embodiment, a program for implementation of a function as the training unit 255 of the processing circuit 250 is stored in the storage circuit 240 in the form of a computer-executable program. For example, the processing circuit 250 is a processor that implements the function of the training unit 255 by reading the program out of the storage circuit 240 and running the read program.

The training unit 255 has a function of acquiring a teacher data group included in a data set stored in the storage circuit 240 for training an image generation model, and training the image generation model. The training unit 255 trains the image generation model by using training data that includes the medical image group described in the first to seventh embodiments, the contrast image group related to the medical image group, and the imaging condition group pertaining to the contrast image group. The imaging condition group mentioned here is an imaging condition group that includes contrast time that includes contrast time moment of at least one point in time. Specifically, when a medical image in the medical image group and contrast time are inputted, by using the training data described above, the training unit 255 trains the image generation model that generates a contrast effect image that depicts a contrast effect corresponding to the contrast time on the basis of the medical image.

The present disclosure makes it possible to desirably acquire an image that depicts a contrast effect corresponding to contrast time that includes contrast time moment of a certain point in time.

### OTHER EMBODIMENTS

An example of an OCTA image of a superficial layer and an FA examination image has been described as images in the field of ophthalmology in the first to sixth embodiments above; however, the scope of the present disclosure is not limited to this configuration. For example, similar processing may be performed using an OCTA image of a choroidal vascular network and an indocyanine green fundus angiography (IA) examination image. Similar processing may be performed using, without being limited to an OCTA image of a choroidal vascular network, an enface image of a choroidal vascular network generated from OCT and an IA examination image.

An example of a CT image and a contrast CT image has been described as images in the field of radiology in the seventh embodiment above; however, the scope of the present disclosure is not limited to this configuration. For example, similar processing may be performed using a contrast CT image of a certain time phase and a contrast CT image of a time phase different from said certain time phase. Similar processing may be performed using images acquired from imaging apparatuses of different types, for example, an MRI image and a contrast CT image.

The contrast effect image outputted by the outputting unit 252 may be processed into an image of another type from which it is possible to know a contrast effect, such as the one described earlier in the second variation example of the third embodiment, and then may be displayed. That is, the contrast effect image outputted by the outputting unit 252 does not have to be displayed on an as-is basis.

The present disclosure may be embodied by supplying, to a system or an apparatus via a network or in the form of a storage medium, a program that realizes one or more functions of the embodiments described above, and by causing one or more processors in the computer of the system or the apparatus to read out and run the program. The present disclosure may be embodied by means of circuitry (for example, ASIC) that realizes the one or more functions.

The program, and a computer-readable storage medium storing the program, are encompassed within the present disclosure.

All of the foregoing embodiments of the present disclosure show just some examples in specific implementation of the present disclosure. The technical scope of the present disclosure shall not be construed restrictively by these examples. That is, the present disclosure can be embodied in various modes without departing from its technical spirit or from its major features.

The embodiments disclosed herein encompass the following configurations, methods, and storage medium.

[Configuration 1] An image generation apparatus comprising: at least one processor; and at least one memory storing instructions, when executed by the at least one processor, causing the image generation apparatus to operate as: an image acquisition unit configured to acquire a medical image; and an outputting unit configured to output a contrast effect image that depicts a contrast effect corresponding to contrast time that includes contrast time moment, the contrast time moment being at least one point in time, based on the medical image acquired by the image acquisition unit, by using an image generation model configured to receive an input of the medical image and generate the contrast effect image depicting the contrast effect.

[Configuration 2] The image generation apparatus according to Configuration 1, wherein the instructions cause the image generation apparatus to further operate as: an imaging condition acquisition unit configured to acquire an imaging condition that includes the contrast time, and, based on the medical image and the imaging condition, the outputting unit outputs the contrast effect image.

[Configuration 3] The image generation apparatus according to Configuration 1 or 2, wherein the outputting unit outputs a moving image comprised of a plurality of contrast effect images each of which is the contrast effect image.

[Configuration 4] The image generation apparatus according to any one of Configurations 1 to 3, wherein the image generation model has a function of receiving an input of the medical image and the contrast time and generating the contrast effect image, and the image generation model is a model having been trained using training data that includes a medical image group pertaining to the medical image, a contrast image group related to the medical image group, and an imaging condition group pertaining to the contrast image group.

[Configuration 5] The image generation apparatus according to Configuration 4, wherein the image generation model is a model having been trained based on a semantic area that is an area in an image included in the training data and is an area that is able to be demarcated in accordance with a manner of depiction in the image or in accordance with information related to the image.

[Configuration 6] The image generation apparatus according to Configuration 4 or 5, wherein the training data includes, as the contrast image group, time-lapse contrast images acquired from an identical target of examination.

[Configuration 7] The image generation apparatus according to any one of Configurations 4 to 6, wherein a medical-image-and-contrast-image pair included in the training data and acquired from an identical target of examination is anatomically aligned.

[Configuration 8] The image generation apparatus according to any one of Configurations 4 to 7, wherein the contrast image group included in the training data includes more contrast images captured in contrast time that includes contrast time moment at which an operator wants to make an observation than contrast images captured in contrast time that includes other contrast time moment.

[Configuration 9] The image generation apparatus according to any one of Configurations 1 to 8, wherein the instructions cause the image generation apparatus to further operate as: an imaging condition acquisition unit configured to acquire imaging conditions that include the contrast time and further include different information other than the contrast time, and the image generation model receives an input of the medical image, the contrast time, and the information other than the contrast time.

[Configuration 10] The image generation apparatus according to Configuration 9, wherein the outputting unit includes a plurality of image generation models each of which is the image generation model, and, based on the information other than the contrast time, the outputting unit selects an appropriate image generation model from among the plurality of image generation models, and, by using the selected image generation model, based on the medical image and the imaging conditions, outputs the contrast effect image.

[Configuration 11] The image generation apparatus according to any one of Configurations 4 to 8, wherein, based on an effective pixel area in the contrast image group included in the training data and acquired from an identical target of examination, the training data is augmented.

[Configuration 12] The image generation apparatus according to any one of Configurations 1 to 11, wherein the medical image is a fundus examination image.

[Configuration 13] The image generation apparatus according to any one of Configurations 1 to 11, wherein the medical image is a radiological image.

[Configuration 14] The image generation apparatus according to any one of Configurations 1 to 13, wherein, based on the medical image, the outputting unit generates a moving image that depicts the contrast effect, and outputs, as the contrast effect image, moving-picture frame images corresponding to the contrast time in the moving image.

[Configuration 15] The image generation apparatus according to any one of Configurations 1 to 14, wherein the instructions cause the image generation apparatus to further operate as: a display unit configured to display the contrast effect image on a display device.

[Configuration 16] An image generation apparatus comprising: at least one processor; and at least one memory storing instructions, when executed by the at least one processor, causing the image generation apparatus to operate as: an image acquisition unit configured to acquire a medical image; and an outputting unit configured to, based on the medical image acquired by the image acquisition unit and contrast time moment, output a contrast effect image that depicts a contrast effect corresponding to the contrast time moment, by using an image generation model configured to receive an input of the medical image and generate the contrast effect image depicting the contrast effect.

[Configuration 17] An image generation apparatus comprising: at least one processor; and at least one memory storing instructions, when executed by the at least one processor, causing the image generation apparatus to operate as: an image acquisition unit configured to acquire a medical image; and an outputting unit configured to, based on the medical image acquired by the image acquisition unit, output a plurality of contrast effect images depicting a contrast effect as a moving image, by using an image generation model configured to receive an input of the medical image and generate the contrast effect image depicting the contrast effect.

[Method 1] An image generation method comprising: acquiring a medical image; and outputting a contrast effect image that depicts a contrast effect corresponding to contrast time that includes contrast time moment, the contrast time moment being at least one point in time, based on the medical image acquired in the acquiring, by using an image generation model configured to receive an input of the medical image and generate the contrast effect image depicting the contrast effect.

[Method 2] An image generation method comprising: acquiring a medical image; and outputting, based on the medical image acquired in the acquiring and contrast time moment, a contrast effect image that depicts a contrast effect corresponding to the contrast time moment, by using an image generation model configured to receive an input of the medical image and generate the contrast effect image depicting the contrast effect.

[Method 3] An image generation method comprising: acquiring a medical image; and outputting, based on the acquired medical image, a plurality of contrast effect images depicting a contrast effect as a moving image, by using an image generation model configured to receive an input of the medical image and generate the contrast effect image depicting the contrast effect.

[Method 4] A training method comprising: training, by using training data that includes a medical image group, a contrast image group related to the medical image group, and an imaging condition group pertaining to the contrast image group and including contrast time including contrast time moment, the contrast time moment being at least one point in time, when a medical image in the medical image group and the contrast time are inputted, based on the medical image, an image generation model configured to generate a contrast effect image that depicts a contrast effect corresponding to the contrast time.

[Medium 1] A non-transitory computer-readable storage medium storing a program causing a computer to function as the units of the image generation apparatus according to any one of Configurations 1 to 17.

The present disclosure is not limited to the embodiments having been described above, and various alterations and modifications can be made without departing from the spirit and scope of the present disclosure. Claims are appended hereto so as to make the claimed scope of the present disclosure public.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

1. An image generation apparatus comprising:
at least one processor; and
at least one memory storing instructions, when executed by the at least one processor, causing the image generation apparatus to operate as:
an image acquisition unit configured to acquire a medical image; and
an outputting unit configured to output a contrast effect image that depicts a contrast effect corresponding to contrast time that includes contrast time moment, the contrast time moment being at least one point in time, based on the medical image acquired by the image acquisition unit, by using an image generation model configured to receive an input of the medical image and generate the contrast effect image depicting the contrast effect.

2. The image generation apparatus according to claim 1, wherein the instructions cause the image generation apparatus to further operate as:
an imaging condition acquisition unit configured to acquire an imaging condition that includes the contrast time, and
based on the medical image and the imaging condition, the outputting unit outputs the contrast effect image.

3. The image generation apparatus according to claim 1, wherein
the outputting unit outputs a moving image comprised of a plurality of contrast effect images each of which is the contrast effect image.

4. The image generation apparatus according to claim 1, wherein
the image generation model has a function of receiving an input of the medical image and the contrast time and generating the contrast effect image, and
the image generation model is a model having been trained using training data that includes a medical image group pertaining to the medical image, a contrast image group related to the medical image group, and an imaging condition group pertaining to the contrast image group.

5. The image generation apparatus according to claim 4, wherein
the image generation model is a model having been trained based on a semantic area that is an area in an image included in the training data and is an area that is able to be demarcated in accordance with a manner of depiction in the image or in accordance with information related to the image.

6. The image generation apparatus according to claim 4, wherein
the training data includes, as the contrast image group, time-lapse contrast images acquired from an identical target of examination.

7. The image generation apparatus according to claim 4, wherein
a medical-image-and-contrast-image pair included in the training data and acquired from an identical target of examination is anatomically aligned.

8. The image generation apparatus according to claim 4, wherein
the contrast image group included in the training data includes more contrast images captured in contrast time that includes contrast time moment at which an operator wants to make an observation than contrast images captured in contrast time that includes other contrast time moment.

9. The image generation apparatus according to claim 1, wherein the instructions cause the image generation apparatus to further operate as:
an imaging condition acquisition unit configured to acquire imaging conditions that include the contrast time and further include different information other than the contrast time, and
the image generation model receives an input of the medical image, the contrast time, and the information other than the contrast time.

10. The image generation apparatus according to claim 9, wherein
the outputting unit includes a plurality of image generation models each of which is the image generation model, and
based on the information other than the contrast time, the outputting unit selects an appropriate image generation model from among the plurality of image generation models, and, by using the selected image generation model, based on the medical image and the imaging conditions, outputs the contrast effect image.

11. The image generation apparatus according to claim 4, wherein
based on an effective pixel area in the contrast image group included in the training data and acquired from an identical target of examination, the training data is augmented.

12. The image generation apparatus according to claim 1, wherein
the medical image is a fundus examination image.

13. The image generation apparatus according to claim 1, wherein
the medical image is a radiological image.

14. The image generation apparatus according to claim 1, wherein
based on the medical image, the outputting unit generates a moving image that depicts the contrast effect, and outputs, as the contrast effect image, moving-picture frame images corresponding to the contrast time in the moving image.

15. The image generation apparatus according to claim 1, wherein the instructions cause the image generation apparatus to further operate as:
a display unit configured to display the contrast effect image on a display device.

16. An image generation apparatus comprising:
at least one processor; and
at least one memory storing instructions, when executed by the at least one processor, causing the image generation apparatus to operate as:
an image acquisition unit configured to acquire a medical image; and
an outputting unit configured to, based on the medical image acquired by the image acquisition unit and contrast time moment, output a contrast effect image that depicts a contrast effect corresponding to the contrast time moment, by using an image generation model configured to receive an input of the medical image and generate the contrast effect image depicting the contrast effect.

17. An image generation apparatus comprising:
at least one processor; and
at least one memory storing instructions, when executed by the at least one processor, causing the image generation apparatus to operate as:
an image acquisition unit configured to acquire a medical image; and
an outputting unit configured to, based on the medical image acquired by the image acquisition unit, output a plurality of contrast effect images depicting a contrast effect as a moving image, by using an image generation model configured to receive an input of the medical image and generate the contrast effect image depicting the contrast effect.

18. An image generation method comprising:
acquiring a medical image; and
outputting a contrast effect image that depicts a contrast effect corresponding to contrast time that includes contrast time moment, the contrast time moment being at least one point in time, based on the medical image acquired in the acquiring, by using an image generation model configured to receive an input of the medical image and generate the contrast effect image depicting the contrast effect.

19. An image generation method comprising:
acquiring a medical image; and
outputting, based on the medical image acquired in the acquiring and contrast time moment, a contrast effect image that depicts a contrast effect corresponding to the contrast time moment, by using an image generation model configured to receive an input of the medical image and generate the contrast effect image depicting the contrast effect.

20. An image generation method comprising:
acquiring a medical image; and
outputting, based on the acquired medical image, a plurality of contrast effect images depicting a contrast effect as a moving image, by using an image generation model configured to receive an input of the medical image and generate the contrast effect image depicting the contrast effect.

21. A training method comprising:
training, by using training data that includes a medical image group, a contrast image group related to the medical image group, and an imaging condition group pertaining to the contrast image group and including contrast time including contrast time moment, the contrast time moment being at least one point in time, when a medical image in the medical image group and the contrast time are inputted, based on the medical image, an image generation model configured to generate a contrast effect image that depicts a contrast effect corresponding to the contrast time.

22. A non-transitory computer-readable storage medium storing a program causing a computer to function as the units of the image generation apparatus according to claim 1.
